SEQUENCE LISTING (36 sequences)

SEQ ID NO: 1 - protein, 13 aa, Artificial Sequence
  PFD5, peptide
PDFDFDFDFDFDP

SEQ ID NO: 2 - protein, 12 aa, Artificial Sequence
  peptide
PEFEFEFEFEFE

SEQ ID NO: 3 - protein, 12 aa, Artificial Sequence
  peptide
EFEFEFEFEFEP

SEQ ID NO: 4 - protein, 13 aa, Artificial Sequence
  peptide
PSFSFSFSFSFSP

SEQ ID NO: 5 - protein, 13 aa, Artificial Sequence
  peptide
  MISC_FEATURE (2)..(2): Phosphoserine
  MISC_FEATURE (4)..(4): Phosphoserine
  MISC_FEATURE (6)..(6): Phosphoserine
  MISC_FEATURE (8)..(8): Phosphoserine
  MISC_FEATURE (10)..(10): Phosphoserine
  MISC_FEATURE (12)..(12): Phosphoserine
PSFSFSFSFSFSP

SEQ ID NO: 6 - protein, 13 aa, Artificial Sequence
  peptide
  MISC_FEATURE (2)..(2): Phosphotyrosine
  MISC_FEATURE (4)..(4): Phosphotyrosine
  MISC_FEATURE (6)..(6): Phosphotyrosine
  MISC_FEATURE (8)..(8): Phosphotyrosine
  MISC_FEATURE (10)..(10): Phosphotyrosine
  MISC_FEATURE (12)..(12): Phosphotyrosine
PYFYFYFYFYFYP

SEQ ID NO: 7 - protein, 13 aa, Artificial Sequence
  peptide
PELELELELELEP

SEQ ID NO: 8 - protein, 13 aa, Artificial Sequence
  peptide
PDLDLDLDLDLDP

SEQ ID NO: 9 - protein, 13 aa, Artificial Sequence
  peptide
PSLSLSLSLSLSP

SEQ ID NO: 10 - protein, 13 aa, Artificial Sequence
  peptide
  MISC_FEATURE (2)..(2): Phosphoserine
  MISC_FEATURE (4)..(4): Phosphoserine
  MISC_FEATURE (6)..(6): Phosphoserine
  MISC_FEATURE (8)..(8): Phosphoserine
  MISC_FEATURE (10)..(10): Phosphoserine
  MISC_FEATURE (12)..(12): Phosphoserine
PSLSLSLSLSLSP

SEQ ID NO: 11 - protein, 13 aa, Artificial Sequence
  peptide
  MISC_FEATURE (2)..(2): Phosphotyrosine
  MISC_FEATURE (4)..(4): Phosphotyrosine
  MISC_FEATURE (6)..(6): Phosphotyrosine
  MISC_FEATURE (8)..(8): Phosphotyrosine
  MISC_FEATURE (10)..(10): Phosphotyrosine
  MISC_FEATURE (12)..(12): Phosphotyrosine
PYLYLYLYLYLYP

SEQ ID NO: 12 - protein, 19 aa, Artificial Sequence
  peptide
PEFSFEFSFEFSFEFSFEP

SEQ ID NO: 13 - protein, 19 aa, Artificial Sequence
  peptide
  MISC_FEATURE (2)..(2): Phosphoserine
  MISC_FEATURE (6)..(6): Phosphoserine
  MISC_FEATURE (10)..(10): Phosphoserine
  MISC_FEATURE (14)..(14): Phosphoserine
PSFSFSFSFSFSFSFSFSP

SEQ ID NO: 14 - protein, 19 aa, Artificial Sequence
  peptide
  MISC_FEATURE (2)..(2): Phosphoserine
  MISC_FEATURE (6)..(6): Phosphoserine
  MISC_FEATURE (10)..(10): Phosphoserine
  MISC_FEATURE (14)..(14): Phosphoserine
PSFEFSFEFSFEFSFEFEP

SEQ ID NO: 15 - protein, 19 aa, Artificial Sequence
  peptide
  MISC_FEATURE (2)..(2): Phosphoserine
  MISC_FEATURE (6)..(6): Phosphoserine
  MISC_FEATURE (10)..(10): Phosphoserine
  MISC_FEATURE (14)..(14): Phosphoserine
PSFDFSFDFSFDFSFDFDP

SEQ ID NO: 16 - protein, 30 aa, Artificial Sequence
  peptide
ALEFEFEFEPAEFEFEFELPALEFEFEFEP

SEQ ID NO: 17 - protein, 13 aa, Artificial Sequence
  peptide
PEFEFEKEFEFEP

SEQ ID NO: 18 - protein, 19 aa, Artificial Sequence
  peptide
PEFEFEFEFEFEGGGRGDS

SEQ ID NO: 19 - protein, 14 aa, Artificial Sequence
  peptide
FEFEFEPGGGRGDS

SEQ ID NO: 20 - protein, 13 aa, Artificial Sequence
  peptide
  MISC_FEATURE (1)..(1): acetylation of the amino terminus
  MISC_FEATURE (13)..(13): amidation of the carboxy terminus
PDFDFDFDFDFDP

SEQ ID NO: 21 - protein, 14 aa, Artificial Sequence
  peptide
PDFDFDFDFDFDFD

SEQ ID NO: 22 - protein, 12 aa, Artificial Sequence
  peptide
FDFDFDFDFDFD

SEQ ID NO: 23 - protein, 13 aa, Artificial Sequence
  peptide
PEFEFEFEFEFEP

SEQ ID NO: 24 - protein, 13 aa, Artificial Sequence
  peptide
  MISC_FEATURE (13)..(13): amidation of the carboxy terminus
PDFDFDFDFDFDP

SEQ ID NO: 25 - protein, 10 aa, Artificial Sequence
  peptide
FEFEFEFEFE

SEQ ID NO: 26 - protein, 12 aa, Artificial Sequence
  peptide
FEFEFEFEFEFE

SEQ ID NO: 27 - protein, 14 aa, Artificial Sequence
  peptide
FEFEFEFEFEFEFE

SEQ ID NO: 28 - protein, 10 aa, Artificial Sequence
  peptide
PDFDFDFDFD

SEQ ID NO: 29 - protein, 14 aa, Artificial Sequence
  peptide
PDFDFDFDFDFDFD

SEQ ID NO: 30 - protein, 18 aa, Artificial Sequence
  peptide
PDFDFDFDFDFDFDFDFD

SEQ ID NO: 31 - protein, 10 aa, Artificial Sequence
  peptide
FDFDFDFDFD

SEQ ID NO: 32 - protein, 12 aa, Artificial Sequence
  peptide
FDFDFDFDFDFD

SEQ ID NO: 33 - protein, 14 aa, Artificial Sequence
  peptide
FDFDFDFDFDFDFD

SEQ ID NO: 34 - protein, 17 aa, Artificial Sequence
  peptide
PDFDFDFDFDFDPRGDS

SEQ ID NO: 35 - protein, 8 aa, Artificial Sequence
  peptide
PFDFDFDP

SEQ ID NO: 36 - protein, 15 aa, Artificial Sequence
  peptide
PFDFDFDPGGGRGDS

(19) United States
(12) Patent Application Publication
(10) Pub. No.: US 2023/0130132 A1
(43) Pub. Date: Apr. 27, 2023

(54) PHARMACEUTICAL COMPOSITIONS COMPRISING AMPHIPHILIC PEPTIDES AND METHODS OF USE THEREOF

(71) Applicant: BONE SCI. BIO LTD., Beer-Sheva (IL)

(72) Inventors: Oron Zeevi-Yacoby, Karmei Yossef (IL); Hanna Rapaport, Lehavim (IL); Noa Harduf, Kibutz Gevim (IL); Dganit Shkedy, Kfar Hess (IL); Hodaya Green, Har-Hebron (IL)

(21) Appl. No.: 17/913,372

(22) PCT Filed: Mar. 24, 2021
(86) PCT No.: PCT/IL2021/050329

§ 371 Date: Sep. 21, 2022

Related U.S. Application Data
(60) Provisional application No. 62/994,904, filed on Mar. 26, 2020.
(60) Provisional application No. 62/994,905, filed on Mar. 26, 2020.

(51) Int. Cl. C07K 9/00; A61K 31/65; A61K 31/43; A61K 47/02

(52) U.S. Cl. CPC C07K 9/00; A61K 31/65; A61K 31/43; A61K 47/02

(57) ABSTRACT

Pharmaceutical compositions comprising amphiphilic peptides are provided. The pharmaceutical compositions are useful in treating or preventing various orthopedic and dental-related infectious and/or inflammatory conditions.

### FIELD OF THE INVENTION

The present invention relates to pharmaceutical compositions comprising at least one amphiphilic peptide, derivative or salt thereof and use thereof.

### BACKGROUND OF THE INVENTION

Amphiphilic peptides, composed of pairs of hydrophobic-hydrophilic alternating amino acid residues self-assemble to form β-sheet structures (Rapaport et al. JACS, 2000, 122:12523-12529; Rapaport et al. JACS, 2002, 124:9342-9343). These structures are characterized by the presence of pleated β-strands that are interlinked by hydrogen bonds and other molecular interactions between amino acid side chains. Amphiphilic peptides that tend to form the β-sheet structure have been shown to assemble into hydrogels suitable for drug delivery as well as for the formation of biocompatible matrices for tissue engineering (Amosi et al. Acta Biomater. 2012, 8:2466-2475; Zarzhitsky et al. J. Coll. Int. Sci. 2011, 360:525-531).

Rapaport et al. (Adv. Func. Mater. 2008, 18(19):2889-2896) describe amphiphilic and acidic β-sheet-forming peptides (AAβPs) having the sequence Pro-Y—(Z—Y)₅—Pro, Y=Glu or Asp and Z=Phe or Leu that may assemble into hydrogel structures at near neutral pH values, several units higher than the intrinsic pKa of their acidic amino acid side chains (Segman-Magidovich et al. Adv. Mater. 2008, 20:2156-2161).

Zarzhitsky et al. (Biopolymers, 2013, 100(6):760-72) describe the effect of peptide concentration, pH, and calcium ions concentration on the self-assembly of amphiphilic and anionic β-sheet peptide hydrogels. The mechanical stability and resistance to dissolution were shown to be highly linked to these factors.

WO 2007/148334 describes amphiphilic peptides comprising predominantly acidic amino acids, which are capable, alone or in combination with ions and minerals, of forming hydrogels at physiological pH and serving as scaffolds for mineralization. The peptides and compositions comprising same are disclosed as being useful for treating diseased or injured bone in orthopedic, periodontal and craniofacial indications.

WO 2009/072119 describes therapeutic uses of amphiphilic peptides and pharmaceutical compositions comprising them for treatment and prevention of progression of osteoporosis and pre-osteoporotic conditions by direct administration into deficient, deteriorated or injured bone and in particular into low bone mineral density sites. The amphiphilic peptides comprise predominantly acidic amino acids, which are capable, alone or in combination with ions and minerals, of forming β-sheet assemblies and hydrogels at physiological pH and serve as scaffolds for mineralization directly at the bone site.

Gretler (Design and Characterization of peptides for Specific Interaction with TiO₂, Ph.D. Thesis, 2011) describes the characterization of the interactions between titanium oxide and peptides decorated with amine, carboxyl and phosophoserine functional groups using analytical liquid chromatography with various loading and eluting solutions.

Gitelman et al. (Langmuir, 2014, 30:4716-4724) describe a bifunctional peptide with a β-strand motif that was shown to strongly bind to titanium dioxide through two phosphorylated serine residues, both situated on the same face of the strand.

Gitelman Povimonsky et al. (J. Mater. Chem. B, 2017, 5:2096-2105) describe peptide coated surfaces that exhibit a larger area of focal adhesion points compared to uncoated TiO₂ surfaces. In the model blood serum, the peptide coated implants were found to adsorb cell adhesion proteins. The efficacy of peptide coated titanium alloy (Ti6Al4V) implants was tested in a rabbit femur bone model. Histology and micro-computerized tomography (mCT) analysis of the removed femora showed a higher bone density and improved bone-implant osseointegration compared to uncoated implants.

U.S. Pat. No. 10,213,527 describes functionalized titanium binding peptides which are capable of promoting bone growth and mineralization.

Drago et al. (Clin. Orthop. Relat. Res., 2014, 472:3311-3323) describe Disposable Antibacterial Coating (DAC) hydrogels as implant coating that reduces in vitro bacterial colonization and biofilm formation.

There remains a yet unmet need for compositions comprising stable hydrogels that can serve as depot systems for drug delivery useful in treating infectious and/or inflammatory diseases, particularly those associated with mineralized tissues and implants.

### SUMMARY OF THE INVENTION

The present invention provides pharmaceutical compositions comprising at least one amphiphilic peptide comprising alternating hydrophobic/hydrophilic amino acid residues, or a derivative or a salt thereof, wherein the hydrophilic amino acid residues are predominantly acidic, and use thereof in treating or preventing infectious and/or inflammatory diseases that are associated with mineralized tissues and implants.

The present invention is based, in part, on the unexpected finding of stable compositions comprising an antibiotic and a peptide having alternating hydrophobic and hydrophilic residues, wherein the hydrophilic residues are predominantly acidic. The compositions can be used as drug delivery depot systems for the treatment or prevention of infections associated with mineralized tissues and implants. Surprisingly, the compositions were shown to enhance natural tissue healing by mimicking negatively charged ECM proteins (bone), provide an effective localized, sustained drug delivery at the target site from several hours to weeks, and afford a strong adherence to the surface of metal, metal oxide, and ceramics implants thereby being an effective drug eluting, protective barrier for orthopedic, dental and other implants. The stable compositions of the present invention are particularly advantageous in providing prolonged anti-bacterial, and/or anti-inflammatory effect thereby affording healing of a soft tissue adjacent to a hard tissue such as a tooth, an implant, and/or a bone. In this manner, tissue repair and regeneration are achieved.

According to one aspect of the present invention, there is provided a pharmaceutical composition comprising:
(i) at least one amphiphilic peptide comprising alternating hydrophobic/hydrophilic amino acid residues, or a derivative or a salt thereof, wherein the peptide comprises 2-20 pairs of hydrophobic-hydrophilic alternating amino acid residues wherein the hydrophilic amino acid residue is selected from the group consisting of a negatively charged amino acid, a hydroxyl-containing amino acid, and a phosphorylated-hydroxyl-containing amino acid, and wherein the peptide has no more than 10% positively charged amino acid residues, and
(ii) an antibiotic selected from the group consisting of a tetracycline, a penicillin, a cephalosporin, an aminoglycozide, a glycopeptide, chloramphenicol, a quinolone, a sulphonamide, 5-nitroimidazole, an ansamycin, a macrolide, and a mixture or combination thereof. Each possibility represents a separate embodiment.

According to one embodiment, the at least one amphiphilic peptide is 4 to 40 amino acids in length, including each integer within the specified range. According to certain embodiments, the at least one amphiphilic peptide is 7 to 28 amino acids in length, including each integer within the specified range. According to additional embodiments, the peptide further comprises at least one terminal Proline (Pro) residue. According to various embodiments, the peptide further comprises two terminal Pro residues. According to some embodiments, the hydrophobic amino acid is selected from the group consisting of Phenylalanine (Phe), Leucine (Leu), Isoleucine (Ile), Valine (Val), Tryptophan (Trp), and Alanine (Ala). Each possibility represents a separate embodiment. According to particular embodiments, the hydrophobic amino acid is Phe or Leu. Each possibility represents a separate embodiment. According to some embodiments, the hydrophilic amino acid is selected from the group consisting of Glutamic acid (Glu), Aspartic acid (Asp), Tyrosine (Tyr), Serine (Ser), Threonine (Thr), Phosphoserine (Ser(PO₄)), Phosphothreonine (Thr(PO₄)), and Phosphotyrosine (Tyr(PO₄)). Each possibility represents a separate embodiment.

According to certain embodiments, the peptide comprises an amino acid sequence as set forth in Formula I:
X-(hydrophobic-hydrophilic)ₙ-B (Formula I) or a pharmaceutically acceptable salt thereof,
wherein n designates an integer from 2 to 20, hydrophobic designates a hydrophobic amino acid residue, hydrophilic designates a hydrophilic amino acid residue, X designates Pro, Pro-hydrophilic or the peptide's amino terminus, and B designates Pro or the peptide's carboxy terminus. Each possibility represents a separate embodiment.

According to various embodiments, the amino terminus is modified. In one embodiment, the amino terminus is acetylated. According to other embodiments, the carboxy terminus is modified. In one embodiment, the carboxy terminus is amidated.

According to particular embodiments, the peptide comprises an amino acid sequence of any of the following formulae X-(Phe-Glu)ₙ-B, X-(Phe-Asp)ₙ-B, X-(Leu-Glu)ₙ-B, and X-(Leu-Asp)ₙ-B or a pharmaceutically acceptable salt thereof, wherein n designates an integer from 2 to 20, X designates Pro, Pro-hydrophilic amino acid residue, or the peptide's amino terminus, and B designates Pro or the peptide's carboxy terminus. Each possibility represents a separate embodiment.

According to specific embodiments, the peptide comprises a sequence selected from the group consisting of:

(SEQ ID NO: 1)
Pro-(Asp-Phe)₅-Asp-Pro,
(SEQ ID NO: 2)
Pro-Glu-(Phe-Glu)₅,
(SEQ ID NO: 3)
Glu-(Phe-Glu)₅-Pro,
(SEQ ID NO: 4)
Pro-(Ser-Phe)₅-Ser-Pro,
(SEQ ID NO: 5)
Pro-(SerPO₄-Phe)₅-SerPO₄-Pro,
(SEQ ID NO: 6)
Pro-(TyrPO₄-Phe)₅-TyrPO₄-Pro,
(SEQ ID NO: 7)
Pro-(Glu-Leu)₅-Glu-Pro,
(SEQ ID NO: 8)
Pro-(Asp-Leu)₅-Asp-Pro,
(SEQ ID NO: 9)
Pro-(Ser-Leu)₅-Ser-Pro,
(SEQ ID NO: 10)
Pro-(SerPO₄-Leu)₅-SerPO₄-Pro,
(SEQ ID NO: 11)
Pro-(TyrPO₄-Leu)₅-TyrPO₄-Pro,
(SEQ ID NO: 12)
Pro-(Glu-Phe-Ser-Phe)₄-Glu-Pro,
(SEQ ID NO: 13)
Pro-(SerPO₄-Phe-Ser-Phe)₄-Ser-Pro,
(SEQ ID NO: 14)
Pro-(SerPO₄-Phe-Glu-Phe)₄-Glu-Pro,
(SEQ ID NO: 15)
Pro-(SerPO₄-Phe-Asp-Phe)₄-Asp-Pro,
(SEQ ID NO: 16)
Ala-Leu-Glu-(Phe-Glu)₃-Pro-Ala-(Glu-Phe)₃-Glu-Leu-
Pro-Ala-Leu-Glu-(Phe-Glu)₃-Pro,
(SEQ ID NO: 17)
Pro-Glu-(Phe-Glu)₂-Lys-(Glu-Phe)₂-Glu-Pro,
(SEQ ID NO: 18)
Pro-Glu-(Phe-Glu)₅-(Gly)₃-Arg-Gly-Asp-Ser,
(SEQ ID NO: 19)
(Phe-Glu)₃-Pro-(Gly)₃-Arg-Gly-Asp-Ser,
(SEQ ID NO: 20)
Ac-Pro-Asp-(Phe-Asp)₅-Pro-NH₂,
(SEQ ID NO: 21)
Pro-Asp-(Phe-Asp)₆,
(SEQ ID NO: 22)
(Phe-Asp)₆,
(SEQ ID NO: 23)
Pro-Glu-(Phe-Glu)₅-Pro,
(SEQ ID NO: 24)
Pro-Asp-(Phe-Asp)₅-Pro-NH₂,
(SEQ ID NO: 25)
(Phe-Glu)₅,
(SEQ ID NO: 26)
(Phe-Glu)₆,
(SEQ ID NO: 27)
(Phe-Glu)₇,
(SEQ ID NO: 28)
Pro-Asp-(Phe-Asp)₄,
(SEQ ID NO: 29)
Pro-Asp-(Phe-Asp)₆,
(SEQ ID NO: 30)
Pro-Asp-(Phe-Asp)₈,
(SEQ ID NO: 31)
(Phe-Asp)₅,
(SEQ ID NO: 32)
(Phe-Asp)₆,
(SEQ ID NO: 33)
(Phe-Asp)₇,
(SEQ ID NO: 34)
Pro-Asp-(Phe-Asp)₅-Pro-Arg-Gly-Asp-Ser,
(SEQ ID NO: 35)
Pro-(Phe-Asp)₃-Pro,
and
(SEQ ID NO: 36)
Pro-(Phe-Asp)₃-Pro-(Gly)₃-Arg-Gly-Asp-Ser,

or a pharmaceutically acceptable salt thereof.

Each possibility represents a separate embodiment.

According to one embodiment, the peptide comprises Pro-(Asp-Phe)₅-Asp-Pro (SEQ ID NO: 1; designated herein “PFD5”). In another embodiment, the peptide comprises the sodium salt of a peptide having a sequence as set forth in SEQ ID NO: 1 (designated herein “PFD5 sodium”). In exemplary embodiments, the peptide or salt thereof is present in the composition in an amount of about 1% to about 5% (w/v), including each value within the specified range.

According to further embodiments, the antibiotic is a tetracycline antibiotic. According to various embodiments, the tetracycline antibiotic is selected from the group consisting of chlortetracycline, oxytetracycline, demeclocycline, doxycycline, lymecycline, meclocycline, methacycline, minocycline, rolitetracycline, chlorotetracycline, tigecycline, and a pharmaceutically acceptable salt thereof. Each possibility represents a separate embodiment. According to specific embodiments, the antibiotic is doxycycline or a pharmaceutically acceptable salt thereof. According to other specific embodiments, the antibiotic is minocycline or a pharmaceutically acceptable salt thereof.

According to further embodiments, the antibiotic is an aminoglycoside. According to various embodiments, the aminoglycoside antibiotic is selected from the group consisting of kanamycin A, amikacin, tobramycin, dibekacin, gentamicin, sisomicin, netilmicin, neomycins B, C or E, streptomycin, and a pharmaceutically acceptable salt thereof. Each possibility represents a separate embodiment. According to particular embodiments, the aminoglycoside is gentamicin or a pharmaceutically acceptable salt thereof.

According to yet other embodiments, the antibiotic is a glycopeptide. According to specific embodiments, the glycopeptide is vancomycin or a pharmaceutically acceptable salt thereof.

According to specific embodiments, the antibiotic is a combination of vancomycin and rifampicin, or pharmaceutically acceptable salts thereof. According to other specific embodiments, the antibiotic is a combination of minocycline and rifampicin, or pharmaceutically acceptable salts thereof.

According to some embodiments, the antibiotic is present in the composition in an amount of about 0.01% to about 20% (w/v), including each value within the specified range. According to particular embodiments, the antibiotic is present in the composition in an amount of about 0.5% to about 10% (w/v), including each value within the specified range.

According to certain embodiments, the pharmaceutical composition further comprises at least one pharmaceutically acceptable excipient. According to some embodiments, the pharmaceutically acceptable excipient comprises at least one of a chelating agent, a buffering or pH adjusting agent, a preservative, an anti-oxidant, a thickening agent, a tonicity enhancing agent, a tissue adhesive, an inorganic mineral, and a combination or mixture thereof. Each possibility represents a separate embodiment.

In various embodiments, the chelating agent is selected from the group consisting of disodium edetate, deferoxamine mesylate (desferrioxamine), 2,3-dimercaprol, meso-2,3-dimercaptosuccinic acid (DMSA) and its ester analogues, deferiprone, nitrilotriacetic acid (NTA), and a mixture or combination thereof. Each possibility represents a separate embodiment.

In other embodiments, the buffering or pH adjusting agent is selected from the group consisting of sodium hydroxide, potassium hydroxide, arginine, lysine, hydrochloric acid, aspartic acid, glutamic acid, and a mixture or combination thereof. Each possibility represents a separate embodiment. In some embodiments, the composition has a pH in the range of about 5 to about 7, including each value within the specified range.

In various embodiments, the preservative comprises at least one of methylparaben, ethylparaben, propylparaben, butylparaben, cresol, chlorocresol, phenolmercuric salt, acetomeroctol, nitromersol, thimerosal, mercurochrome, mercuric salt, silver, silver salt, copper, copper salt, sodium salt, potassium salt, hydroquinone, pyrocatechol, resorcinol, 4-n-hexyl resorcinol, benzalkonium chloride, benzethonium chloride, benzoic acid, benzyl alcohol, cetylpyridinium chloride, chlorobutanol, dehydro acetic acid, o-phenylphenol, phenol, phenyl ethyl alcohol, sorbic acid, thimerosal, thymol, phenylmercuric salt, formaldehyde, and a mixture or combination thereof. Each possibility represents a separate embodiment.

In further embodiments, the anti-oxidant comprises at least one of ascorbic acid, N-acetyl cysteine (NAC), derivatives and salts thereof. Each possibility represents a separate embodiment. In particular embodiments, the NAC or a derivative or salt thereof comprises at least one of N-acetylcysteine, S-(2-(1-carboxy-2-methylpropyl)isoindole-1-yl)-N-acetylcysteine, N-acetylcysteine lysinate, S-phenyl-N-acetylcysteine, N-acetyl-S-(N-methylcarbamoyl)cysteine, N-acetyl-S-pentachloro-1,3-butadienylcysteine, adamantyl-N-acetylcystein, N-acetylcysteinamide, S-(1-(4′-methoxyphenyl)-2-hydroxypropyl)-N-acetylcysteine, S-(N,N-diethyldithiocarbamoyl)-N-acetylcysteine, S-(6-purinyl)-N-acetylcysteine, S-(3-oxopropyl)-N-acetylcysteine, S-(2-carboxyethyl)-N-acetylcysteine, S-(3-hydroxy-3-carboxy-n-propyl)-N-acetylcysteine, N-acetylcysteine-6,7-dihydro-7-hydroxy-1-hydroxymethyl-5H-pyrrolizine, S-(2-(N(7)-guanyl)ethyl)-N-acetylcysteine, S-trichlorovinyl-N-acetylcysteine, S-(2-methylbenzyl)-N-acetylcysteine, S-1,2-dichlorovinyl-N-acetylcysteine, S-(2-((1-methylethyl)phenylamino)-2-oxoethyl)-L-cysteine, S-nitro so-N-acetylcysteine, and a mixture or combination thereof. Each possibility represents a separate embodiment.

In other embodiments, the thickening agent comprises at least one of hydroxypropyl methylcellulose (HPMC), hydroxypropyl cellulose (HPC), hydroxyethyl cellulose (HEC), hydroxymethyl cellulose (HMC), carboxy methyl cellulose (CMC), polyvinylpyrrolidone (PVP), polyvinyl alcohol (PVA), and a mixture or combination thereof. Each possibility represents a separate embodiment.

In certain embodiments, the tonicity enhancing agent comprises at least one of mannitol, glycerol, sorbitol, xylitol, and a mixture or combination thereof. Each possibility represents a separate embodiment.

In various embodiments, the tissue adhesive comprises at least one of fibrin(ogen), gelatin, collagen, chitosan, cyanoacrylate, and a mixture or combination thereof. Each possibility represents a separate embodiment.

In some embodiments, the inorganic mineral comprises at least one of hydroxyapatite, calcium phosphate, calcium carbonate, calcium gluconate, calcium oxalate, calcium sulfate, calcium chloride, magnesium phosphate, magnesium carbonate, magnesium gluconate, magnesium oxalate, magnesium sulfate, magnesium chloride, zinc phosphate, zinc carbonate, zinc gluconate, zinc oxalate, zinc sulfate, zinc chloride, sodium bicarbonate, and a mixture or combination thereof. Each possibility represents a separate embodiment. In currently preferred embodiments, the inorganic mineral is a calcium phosphate mineral selected from the group consisting of amorphous calcium phosphate, tricalcium phosphate, β-tri-calcium phosphate, hydroxyapatite, and a mixture or combination thereof. Each possibility represents a separate embodiment.

In additional embodiments, the composition disclosed herein further comprises at least one of an antiresorptive agent and an anti-inflammatory agent. Each possibility represents a separate embodiment.

In further embodiments, the antiresorptive agent is a bisphosphonate selected from the group consisting of zoledronic acid, pamidronate, alendronate, etidronate, clodronate, risedronate, tiludronate, ibandronate, incadronate, minodronate, olpadronate, neridronate, and EB-1053, or a pharmaceutically acceptable salt thereof. Each possibility represents a separate embodiment.

In additional embodiments, the anti-inflammatory agent is a non-steroidal anti-inflammatory drug (NSAID). In one embodiment, the NSAID is selected from the group consisting of COX-2 inhibitors, sulphonamides, ibuprofen, flurbiprofen, diclofenac, and naproxen, or a pharmaceutically acceptable salt thereof. Each possibility represents a separate embodiment.

According to further embodiments, the composition disclosed herein is in a liquid, semi-solid, or solid form. Each possibility represents a separate embodiment. In other embodiments, the composition is in a form selected from the group consisting of a hydrogel, a solution, a putty, a paste, an emulsion, a suspension, and a powder. Each possibility represents a separate embodiment. In currently preferred embodiments, the composition is in the form of a hydrogel.

According to certain embodiments, the composition disclosed herein is useful in treating or preventing a disease or disorder associated with mineralized tissue. In some embodiments, there is provided a method of treating or preventing a disease or disorder selected from the group consisting of a subchondral bone lesion, osteomyelitis, peri-prosthetic joint infection, and surgery site infection, the method comprising the step of administering to a subject in need thereof a therapeutically effective amount of a composition as disclosed herein. Each possibility represents a separate embodiment.

In various embodiments, there is provided a method of treating or preventing an infection in a subject having a primary joint replacement, a revision joint replacement, avascular necrosis, a high risk contralateral hip fracture, a delayed union fracture, a non-union fracture, an open fracture, a cartilage transplant, or a stress fracture, the method comprising the step of administering to a subject in need thereof a therapeutically effective amount of a composition as disclosed herein. Each possibility represents a separate embodiment.

In some embodiments, there is provided a method of inducing bone repair and regeneration, the method comprising the step of administering to a subject in need thereof a therapeutically effective amount of a composition as disclosed herein.

In additional embodiments, there is provided a method of treating bone fractures selected from the group consisting of stress fractures, delayed union or non-union fractures, and high-risk contralateral hip fracture or infections associated therewith, the method comprising the step of administering to a subject in need thereof a therapeutically effective amount of a composition as disclosed herein. Each possibility represents a separate embodiment.

In further embodiments, there is provided a method of treating a bacterial infection, the method comprising the step of administering to a subject in need thereof a therapeutically effective amount of a composition as disclosed herein. In one embodiment, the bacterial infection is in proximity to mineralized tissue.

In other embodiments, there is provided a method of treating inflammation, the method comprising the step of administering to a subject in need thereof a therapeutically effective amount of a composition as disclosed herein. In one embodiment, the inflammation is in proximity to mineralized tissue.

In particular embodiments, there is provided a method of treating osteoarthritis, the method comprising the step of administering to a subject in need thereof a therapeutically effective amount of a composition as disclosed herein.

In specific embodiments, there is provided a method of treating or preventing a disease or disorder selected from the group consisting of jaw osteonecrosis and peri-mucositis, the method comprising the step of administering to a subject in need thereof a therapeutically effective amount of a composition as disclosed herein. Each possibility represents a separate embodiment.

In other embodiments, there is provided a method of treating or preventing a progressive periodontal disease, the method comprising the step of administering to a subject in need thereof a therapeutically effective amount of a composition as disclosed herein. In some embodiments, the progressive periodontal disease is selected from the group consisting of periodontitis and periimplantitis. Each possibility represents a separate embodiment. In one embodiment, there is provided a method of treating periimplantitis, the method comprising the step of administering to a subject in need thereof a therapeutically effective amount of a composition as disclosed herein.

In various embodiments, there is provided a method of reducing or suppressing bone loss supporting a tooth or a peri-implant, the method comprising the step of administering to a subject in need thereof a therapeutically effective amount of a composition as disclosed herein.

In other embodiments, there is provided a method of inducing bone repair and regeneration in proximity to a tooth or a peri-implant, the method comprising the step of administering to a subject in need thereof a therapeutically effective amount of a composition as disclosed herein.

In further embodiments, there is provided a method of reducing the depth of a periodontal pocket around a tooth or a peri-implant, the method comprising the step of administering to a subject in need thereof a therapeutically effective amount of a composition as disclosed herein. In various embodiments, the composition is applied to a peri-implant. In other embodiments, the composition is administered in proximity to a tooth or a peri-implant via injection to the soft tissue surrounding a tooth or a peri-implant. In yet other embodiments, the composition is topically spread in an open crevice (for example a periodontal pocket) in proximity to a tooth or a peri-implant.

In several embodiments, there is provided a method of coating an implant, the method comprising the step of applying a therapeutically effective amount of a composition as disclosed herein to the implant prior to implantation. In some embodiments, the implant is a metal implant, a metal oxide implant or a ceramic implant. Each possibility represents a separate embodiment.

In additional embodiments, there is provided a method of treating or preventing an implant-related infection, the method comprising the step of administering to a subject in need thereof a therapeutically effective amount of a composition as disclosed herein.

In various embodiments, administration is performed via a route selected from the group consisting of topical, transdermal, intra-lesion, intra-uterine, intra-bladder, intra-ocular, intra-auricular, intra-articular, intra-body cavities, intra-muscular, intra-cutaneous, intra-osseous, subchondral, and subcutaneous. Each possibility represents a separate embodiment.

According to another aspect of the present invention, there is provided a composition comprising:
(i) at least one amphiphilic peptide comprising alternating hydrophobic/hydrophilic amino acid residues, or a derivative or a salt thereof, wherein the peptide comprises 2-20 pairs of hydrophobic-hydrophilic alternating amino acid residues wherein the hydrophilic amino acid residue is selected from the group consisting of a negatively charged amino acid, a hydroxyl-containing amino acid, and a phosphorylated-hydroxyl-containing amino acid, and wherein the peptide has no more than 10% positively charged amino acid residues, and
(ii) N-acetyl cysteine (NAC), derivatives and salts thereof.

According to yet another aspect of the present invention, there is provided a method of treating osteoarthritis, the method comprising the step of intraarticularly administering to a subject in need thereof a therapeutically effective amount of a pharmaceutical composition comprising at least one amphiphilic peptide comprising alternating hydrophobic/hydrophilic amino acid residues, or a derivative or a salt thereof, wherein the peptide comprises 2-20 pairs of hydrophobic-hydrophilic alternating amino acid residues wherein the hydrophilic amino acid residue is selected from the group consisting of a negatively charged amino acid, a hydroxyl-containing amino acid, and a phosphorylated-hydroxyl-containing amino acid, and wherein the peptide has no more than 10% positively charged amino acid residues. In one embodiment, the at least one amphiphilic peptide is present in the composition in an amount of about 1% to about 5% (w/v).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

FIG. 1A-1B. (1A) Images of compositions prepared with different peptide concentrations and peptide to acid mole ratios as outlined in Table 2. Upper row corresponds to upright vials and lower row corresponds to inverted vials. (1B) Images of a composition prepared with 4.8% peptide and 48 mM HCl at a peptide to acid mole ratio of 1:1.67.

FIG. 2A-2B. (2A) An image of a hydrogel of formulation #14. (2B) An image of a putty formed by supplementing formulation #14 with TCP and CMC.

FIG. 3. Images of hydrogels of formulations #14-#20a as outlined in Table 3.

FIG. 4. Images of a putty formed by supplementing formulation #17 with 30 mg CMC and 2.3 g TCP.

FIG. 5. Images of hydrogels of formulations #21-#24 showing vials after reconstitution in 1 ml in upright position (left) and inverted position (right).

FIG. 6. Images of putties of formulations #25-#26.

FIG. 7A-7B. (7A) Images of emulsion-like solutions of formulations #29, #30 and #30a containing, from left to right, 5%, 3.75% and 2.5% peptide, respectively before being supplemented with TCP. (7B) Images of putties of formulations #29, #30 and #30a showing putties formed after mixing with 140% w/v TCP.

FIG. 8. In-vitro release profiles of minocycline from formulation #43 to a 100 ml (▴) and 250 ml (♦) buffer solutions vs. the release profile of minocycline from a Minocin®-like solution (▪).

FIG. 9A-9B. Images indicating the area of local administration of a composition according to embodiments of the present invention with/without teeth.

FIG. 10 demonstrates the strength of a hydrogel, according to resistance to flow in a flipped over vial, formed with and without the presence of NAC.

FIG. 11 demonstrates the oxidation of tetracycline antibiotics with and without the presence of NAC after 4 days at room temperatures.

FIG. 12A-12B show the rheological properties of a hydrogel formed with and without the presence of NAC where the upper curves correspond to hydrogels containing 0.8% NAC and the lower curves correspond to hydrogels which are devoid of NAC.

FIG. 13A-13B demonstrate the strength of hydrogels, according to resistance to flow in a flipped over vial, containing different concentrations of PFDS peptide with (13B) and without (13A) the presence of 0.8% NAC.

FIG. 14A-14H show images of hydrogels on Ti discs. (14A) PFDS hydrogel and vancomycin at t=0; (14B) PFDS hydrogel and vancomycin at t=2 days; (14C) PFDS hydrogel at t=0; (14D) PFDS hydrogel at t=7 days; (14E) PFDS hydrogel at t=0; (14F) PFDS hydrogel at t=1 day; (14G) hyaluronic acid (same concentration as PFDS) hydrogel at t=0; (14H) hyaluronic acid hydrogel at t=1 hour.

FIG. 15 shows the release of vancomycin from a 50-100 mg PFDS hydrogel placed on a titanium disc.

FIG. 16A-16J show images of hydrogels on Co Cr cylinders. (16A) PFD5 hydrogel and vancomycin at t=0; (16B) PFD5 hydrogel and vancomycin at t=3 days; (16C) PFD5 hydrogel at t=0; (16D) PFD5 hydrogel upon insertion to the buffer; (16E) PFD5 hydrogel after 1 day in buffer; (16F) cylinder coated with PFD5 hydrogel withdrawn from buffer after 1 day; (16G) hyaluronic acid hydrogel at t=0; (16H) hyaluronic acid hydrogel upon insertion to the buffer; (16I) hyaluronic acid hydrogel after 1 hour in buffer; (16J) cylinder coated with hyaluronic acid hydrogel withdrawn from buffer after 1 hour.

FIG. 17 shows the release of vancomycin from a PFD5 hydrogel on Co Cr cylinder.

FIG. 18A-18B show adherence of PFD5 hydrogel with vancomycin to a stainless steel implant. (18A) PFD5 hydrogel at t=0; (18B) PFD5 hydrogel after 3 days incubation in a buffer.

FIG. 19A-19B show adherence of PFD5 hydrogel to a stainless steel implant. (19A) PFD5 hydrogel at t=0; (19B) PFD5 hydrogel after 1 day incubation in a buffer.

FIG. 20A-20B show hyaluronic acid hydrogel on a stainless steel implant. (20A) hyaluronic acid hydrogel at t=0; (20B) hyaluronic acid hydrogel after 1 hour incubation in a buffer.

### DETAILED DESCRIPTION OF THE INVENTION

The present invention relates to pharmaceutical compositions comprising amphiphilic peptides and salts or derivatives thereof together with an antibiotic. The compositions are useful for treating or preventing various diseases and disorders that are associated with mineralized tissue by providing sustained, high dose local delivery of the antibiotics incorporated therein over a period of several days.

The treatment of diseases or disorders associated with mineralized tissue according to the principles of the present invention comprises at least one of the following:
an anti-bacterial treatment aimed at inhibiting pathogenic bacteria replication with reduction of bacterial biofilm formation, load or even complete eradication of the bacteria upon treatment.
an anti-inflammatory treatment aimed at reducing or eliminating the inflammatory lesion around the infected hard tissue thereby attenuating disease progression.
an enhancement of bone repair and regeneration by inducing and/or enhancing biomineralization.

The beneficial effects of the composition disclosed herein may be attributed to at least one of the following:
the formation of scaffolds that enhance natural tissue healing by mimicking negatively charged ECM proteins (bone), glycosaminoglycans, and fibronectin (soft tissue).
the entrapment of antibiotics due to electrostatic and hydrophobic interactions thereby enabling an effective, localized, sustained drug delivery at the target site from several hours to weeks.
The strong adhesion of hydrogels to implant surfaces, thereby enabling an effective drug eluting, protective barrier which prevents biofilm formation and recolonization for orthopedic, dental and other implants.

The principles and operation of the present invention may be better understood with reference to the accompanying descriptions. Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Periodontal diseases are inflammatory conditions affecting the tissues surrounding a tooth or a dental implant. While at the early stages, called gingivitis or mucositis, infection only affects the soft tissues surrounding a tooth or an implant, respectively, the more progressive stages, called periodontitis or periimplantitis, are characterized by loss of tooth- or implant-supporting bone, respectively. Treatment of early stages typically includes utilization of different manual ablations, laser-supported systems as well as photodynamic therapy, which may be extended by local or systemic antibiotics. The aforementioned treatments are in most cases insufficient to afford recovery of the periodontal tissue at progressive disease stages thereby requiring surgical procedures. Accordingly, periodontitis and periimplantitis remain as major public health problems leading to loss of teeth and dental implants as well as other complications.

The present invention is based, in part, on the surprising discovery of compositions which effectively prevent and treat progressive periodontal diseases thereby obviating the need for invasive surgical procedures.

An implant-related infection (IRI) is considered one of the most severe complications in orthopedics. Implications thereof include implant failure, osteomyelitis, amputation and even mortality. Periprosthetic Joint Infection (PJI) following hip/knee replacement is a life threatening condition. Current standard of care includes prolonged, mega dose systemic antibiotics with limited efficacy due to difficulties in reaching therapeutically effective local drug concentrations. A two stage procedure is often performed. The first stage includes implanting an antibiotic eluting local delivery device composed of poly(methyl methacrylate) bone cement. The second stage includes removal of the PMMA in a second invasive procedure which possesses the risk of antibiotic resistance and damage to bone tissue.

The present invention provides compositions which were shown to strongly adhere to metal, metal oxides, and ceramics implants thereby forming a coating barrier that prevents biofilm formation and bacteria recolonization extending for weeks. The compositions are loaded with antibiotics providing a sustained release depot system which releases a local therapeutically effective amount of the antibiotic over a period of days. The compositions of the present invention are therefore advantageous in treating and reducing the occurrence of infections in subjects having orthopedic, dental and other implants.

The compositions disclosed herein are useful in treating diseases or disorders which are associated with mineralized tissues including various orthopedic, and dental infections and/or inflammatory conditions. The compositions comprise antibiotics in combination with peptides comprising alternating hydrophobic/hydrophilic amino acid residues, in which the hydrophilic amino acid residues are predominantly acidic. The compositions can further be supplemented with pH adjusting agents such as proton donors or acceptors, thickening agents, inorganic minerals, anti-oxidants, and/or tissue adhesives. The compositions may further comprise anti-resorptive agents to provide enhanced bone resorption capacity thereby suppressing bone loss and inducing bone repair and regeneration and/or anti-inflammatory agents. As demonstrated herein, the compositions are capable of forming stable and homogenous emulsions and hydrogels characterized by superior stability and consistency as well as improved rheological properties. Supplementing the emulsions or hydrogels with minerals provides putties which are particularly useful in inducing bone regeneration.

Further provided herein is a composition comprising at least one amphiphilic peptide comprising alternating hydrophobic/hydrophilic amino acid residues, or a derivative or a salt thereof, wherein the peptide comprises 2-20 pairs of hydrophobic-hydrophilic alternating amino acid residues wherein the hydrophilic amino acid residue is selected from the group consisting of a negatively charged amino acid, a hydroxyl-containing amino acid, and a phosphorylated-hydroxyl-containing amino acid, and wherein the peptide has no more than 10% positively charged amino acid residues; and N-acetyl cysteine (NAC), derivatives and salts thereof. The compositions may further comprise an active agent such as, but not limited to, antibiotics, anti-inflammatory agents, anti-resorptive agents, and chemotherapeutic agents. Each possibility represents a separate embodiment. Without being bound by any theory or mechanism of action, the presence of NAC stabilizes the hydrogel such that it exhibits superior rheological properties with elastic modulus higher than a comparable hydrogel that does not include NAC. These compositions are useful in treating infectious and/or inflammatory diseases, particularly those associated with mineralized tissues and implants.

Within the scope of the present invention is a pharmaceutical composition comprising at least one amphiphilic peptide comprising alternating hydrophobic/hydrophilic amino acid residues, or a derivative or a salt thereof, wherein the peptide comprises 2-20 pairs of hydrophobic-hydrophilic alternating amino acid residues wherein the hydrophilic amino acid residue is selected from the group consisting of a negatively charged amino acid, a hydroxyl-containing amino acid, and a phosphorylated-hydroxyl-containing amino acid, and wherein said peptide has no more than 10% positively charged amino acid residues, the pharmaceutical composition is formulated for intra-articular administration for use in treating osteoarthritis.

Pharmaceutical Compositions

According to certain aspects and embodiments, there is provided a pharmaceutical composition comprising a peptide or derivatives or salts thereof comprising 2 to 20 pairs of hydrophobic-hydrophilic alternating amino acid residues, wherein the hydrophilic amino acid residue is selected from the group consisting of: a negatively charged amino acid, a hydroxyl-containing amino acid, and a phosphorylated-hydroxyl-containing amino acid, and wherein said peptide has no more than 10% positively charged amino acid residues. Typical peptide lengths within the scope of the present invention include, but are not limited to, 4 to 40 amino acids, including each integer within the specified range. In other embodiments, the length of the peptide is about 7 to about 28 amino acids, about 9 to about 20 amino acids, or about 11 to about 18 amino acids, including each integer within the specified ranges. For example, the pharmaceutical composition of the present invention encompasses peptides having 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids, with each possibility representing a separate embodiment.

Also included within the scope of the present invention are salts and derivatives of the peptides used in the disclosed compositions and methods.

As used herein, the term “salts” refers to salts of carboxyl groups also termed base addition salts and to acid addition salts of amino or guanidino groups of the peptide molecule. Suitable base addition salts include, but are not limited to, metallic salts of calcium, lithium, magnesium, potassium, sodium, aluminum, ferric and zinc; ammonium salts derived from ammonia, primary, secondary, tertiary and quaternary amines, non-limiting examples of which are trimethylamine, cyclohexylamine, benzylamine, dibenzylamine, 2-hydroxyethylamine, bis(2-hydroxyethyl)amine, phenylethylbenzylamine, dibenzylethylenediamine, procaine, chloroprocaine, piperidine, monoethanolamine, triethanolamine, quinine, choline, and N-methylglucosamine. Each possibility represents a separate embodiment. Salts with amino acids such as glycine, ornithine, histidine, phenylglycine, lysine, and arginine are contemplated. Each possibility represents a separate embodiment. Furthermore, any zwitterionic salts formed by a carboxylic acid and an amino or guanidino groups of the peptide molecule are contemplated as well.

Suitable acid addition salts include salts derived from inorganic acids such as, but not limited to, hydrochloric, nitric, phosphoric, sulfuric, hydrobromic, hydriodic, phosphorous, and the like, as well as salts derived from organic acids such as aliphatic mono- and dicarboxylic acids such as acetic acid or oxalic acid, phenyl-substituted alkanoic acids, hydroxy alkanoic acids, alkanedioic acids, aromatic acids, aliphatic and aromatic sulfonic acids and the like. Each possibility represents a separate embodiment. The salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Each possibility represents a separate embodiment. Also contemplated are salts of amino acids such as arginate and the like and gluconate or galacturonate. Each possibility represents a separate embodiment.

The acid addition salts may be prepared by known methods of the art in which the free base form is brought into contact with a sufficient amount of the desired acid to produce the salt. The base addition salts are prepared by known methods of the art in which the free acid form is brought into contact with a sufficient amount of the desired base to produce the salt.

“Derivatives” of the peptides of the invention as used herein cover derivatives which may be prepared from the functional groups which occur as side chains on the residues or the N- or C-terminal groups, by means known in the art, and are included in the invention provided that they do not adversely affect the therapeutic benefits of the compositions containing them and do not confer toxic properties to said compositions.

These derivatives may include, for example, aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or aroyl groups), or O-acyl derivatives of free hydroxyl group (e.g., that of seryl or threonyl residues) formed by reaction with acyl moieties. Additional derivatives within the scope of the present invention include peptides formed by phosphorylation of hydroxyl-containing amino acids. It is contemplated that the amino acid residues of the present invention include both D- and L-amino acids, preferably L-amino acids.

As used herein, the term “a hydrophobic amino acid residue” refers to the following amino acids: Alanine (Ala; A), Isoleucine (Ile; I), Leucine (Leu; L), Phenylalanine (Phe; F), Valine (Val; V), and Tryptophan (Trp; W). Each possibility represents a separate embodiment. Currently preferred are Phenylalanine (Phe; F) and Leucine (Leu; L). Each possibility represents a separate embodiment.

As used herein, the term “a hydrophilic amino acid residue” refers to the following amino acids: Glutamic acid (Glu; E), Aspartic acid (Asp; D), Tyrosine (Tyr; Y), Serine (Ser; S), Threonine (Thr; T), Phosphoserine (Ser(PO₄); S(PO₄)), Phosphothreonine (Thr(PO₄); T(PO₄)), and Phosphotyrosine (Tyr(PO₄); Y(PO₄)). Each possibility represents a separate embodiment.

According to additional embodiments, the peptide further comprises at least one terminal Proline (Pro) residue. According to various embodiments, the peptide further comprises two terminal Pro residues.

According to certain aspects and embodiments, the peptide comprises an amino acid sequence represented by the following Formula I:

X-(hydrophobic-hydrophilic)ₙ-B (Formula I) or a pharmaceutically acceptable salt thereof with the following designations:
n is an integer from 2 to 20, including each integer within the specified range;
hydrophobic is a hydrophobic amino acid residue as defined hereinabove;
hydrophilic is a hydrophilic amino acid residue as defined hereinabove;
X is Pro, Pro-hydrophilic or the peptide's amino terminus; and
B is Pro or the peptide's carboxy terminus.

In various aspects and embodiments, the peptide of Formula I is selected from the group consisting of X-(Phe-Glu)ₙ-B, X-(Phe-Asp)ₙ-B, X-(Leu-Glu)ₙ-B, and X-(Leu-Asp)ₙ-B or a pharmaceutically acceptable salt thereof, wherein n, X, and B are as defined hereinabove. Each possibility represents a separate embodiment.

Non-limiting examples of peptides within the scope of the present invention are listed in Table 1 below:

TABLE 1

| SEQ | |
|---|---|
| ID NO: | Sequence |
| 1 | Pro-(Asp-Phe)₅-Asp-Pro |
| 2 | Pro-Glu-(Phe-Glu)₅ |
| 3 | Glu-(Phe-Glu)₅-Pro |
| 4 | Pro-(Ser-Phe)₅-Ser-Pro |
| 5 | Pro-(SerPO₄-Phe)₅-SerPO₄-Pro |
| 6 | Pro-(TyrPO₄-Phe)₅-TyrPO₄-Pro |
| 7 | Pro-(Glu-Leu)₅-Glu-Pro |
| 8 | Pro-(Asp-Leu)₅-Asp-Pro |
| 9 | Pro-(Ser-Leu)₅-Ser-Pro |
| 10 | Pro-(SerPO₄-Leu)₅-SerPO₄-Pro |
| 11 | Pro-(TyrPO₄-Leu)₅-TyrPO₄-Pro |
| 12 | Pro-(Glu-Phe-Ser-Phe)₄-Glu-Pro |
| 13 | Pro-(SerPO₄-Phe-Ser-Phe)₄-Ser-Pro |
| 14 | Pro-(SerPO₄-Phe-Glu-Phe)₄-Glu-Pro |
| 15 | Pro-(SerPO₄-Phe-Asp-Phe)₄-Asp-Pro |
| 16 | Ala-Leu-Glu-(Phe-Glu)₃-Pro-Ala-(Glu-Phe)₃- |
| | Glu-Leu-Pro-Ala-Leu-Glu-(Phe-Glu)₃-Pro |
| 17 | Pro-Glu-(Phe-Glu)₂-Lys-(Glu-Phe)₂-Glu-Pro |
| 18 | Pro-Glu-(Phe-Glu)₅-(Gly)₃-Arg-Gly-Asp-Ser |
| 19 | (Phe-Glu)₃-Pro-(Gly)₃-Arg-Gly-Asp-Ser |
| 20 | Ac-Pro-Asp-(Phe-Asp)₅-Pro-NH₂ |
| 21 | Pro-Asp-(Phe-Asp)₆ |
| 22 | (Phe-Asp)₆ |
| 23 | Pro-Glu-(Phe-Glu)₅-Pro |
| 24 | Pro-Asp-(Phe-Asp)₅-Pro-NH₂ |
| 25 | (Phe-Glu)₅ |
| 26 | (Phe-Glu)₆ |
| 27 | (Phe-Glu)₇ |
| 28 | Pro-Asp-(Phe-Asp)₄ |
| 29 | Pro-Asp-(Phe-Asp)₆ |
| 30 | Pro-Asp-(Phe-Asp)₈ |
| 31 | (Phe-Asp)₅ |
| 32 | (Phe-Asp)₆ |
| 33 | (Phe-Asp)₇ |
| 34 | Pro-Asp-(Phe-Asp)₅-Pro-Arg-Gly-Asp-Ser |
| 35 | Pro-(Phe-Asp)₃-Pro |
| 36 | Pro-(Phe-Asp)₃-Pro-(Gly)₃-Arg-Gly-Asp-Ser |

The peptides, derivatives and salts used in the compositions and methods of the present invention may be synthesized using any method known in the art including, but not limited to, solid phase and liquid phase peptide synthesis. In some embodiments, the peptides, are synthesized using conventional synthesis techniques, e.g., by chemical synthesis techniques. These methods include exclusive solid phase synthesis, partial solid phase synthesis methods, fragment condensation, and classical solution synthesis. Solid phase peptide synthesis procedures are well known in the art and described for example by Stewart & Young, Solid Phase Peptide Syntheses, Pierce Chemical Company, 1984, 2^{nd} Ed. A skilled artesian may synthesize any of the peptides of the present invention by using an automated peptide synthesizer using standard chemistry such as, for example, t-Boc or Fmoc chemistry. Synthetic peptides can be purified by preparative high-performance liquid chromatography (HPLC) as known in the art and their sequences can be confirmed via amino acid sequencing.

Alternatively, the peptides may be prepared by known recombinant DNA techniques by cloning and expressing within a host microorganism or cell a DNA fragment carrying a coding sequence of the selected peptide or construct. Such techniques were described for example, by Bitter et al. Met. Enzymol. 1987, 153:516-544; Studier et al. Met. Enzymol. 1990, 185:60-89; Brisson et al. Nature, 1984, 310:511-514; Takamatsu et al. EMBO J. 1987, 6:307-311; Coruzzi et al. EMBO J. 1984, 3:1671-1680; Brogli et al. Sci. 1984, 224:838-843, Gurley et al. Mol. Cell. Biol. 1986, 6:559-565; and Weissbach & Weissbach, Met. Plant Mol. Biol. 1988, Academic Press, NY, VIII: 421-463. Coding sequences for the peptides can be prepared synthetically, or can be derived from viral RNA by known techniques, or from available cDNA-containing plasmids.

In some aspects and embodiments, the peptide or derivative or salt thereof is present in the composition in an amount of from about 0.2% to about 20% (w/v) of the total weight of the composition, including each value within the specified range. Typically, the amount of the peptide or derivative or salt thereof in the composition is in the range of about 0.5% to about 10% (w/v) of the total weight of the composition, including each value within the specified range. For example, the amount of the peptide or derivative or salt thereof in the composition is about 0.2%, about 0.5%, about 0.7%, about 1%, about 1.5%, about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, or about 20% (w/v) of the total weight of the composition, with each possibility representing a separate embodiment. Currently preferred amount of the peptide or derivative or salt thereof in the composition is from about 1% to about 5% (w/v), including each value within the specified range.

According to certain aspects and embodiments, the composition comprises at least one antibiotics selected from the group consisting of a tetracycline, a penicillin, a cephalosporin, an aminoglycozide, a glycopeptide, chloramphenicol, a quinolone, a sulphonamide, 5-nitroimidazole, an ansamycin, a macrolide, and a mixture or combination thereof. Each possibility represents a separate embodiment.

Suitable tetracycline antibiotics within the scope of the present invention include, but are not limited to, naturally-occurring tetracyclines such as chlortetracycline, oxytetracycline and demeclocycline; and semi-synthetic tetracyclines such as doxycycline, lymecycline, meclocycline, methacycline, minocycline, rolitetracycline, chlorotetracycline, and tigecycline, or pharmaceutically acceptable salts thereof. Each possibility represents a separate embodiment. Currently preferred is the use of doxycycline or a pharmaceutically acceptable salt thereof (e.g. doxycycline hyclate) or the use of minocycline or a pharmaceutically acceptable salt thereof (e.g. minocycline hydrochloride).

Suitable aminoglycoside antibiotics within the scope of the present invention include, but are not limited to, kanamycin A, amikacin, tobramycin, dibekacin, gentamicin, sisomicin, netilmicin, neomycins B, C or E, streptomycin, and a pharmaceutically acceptable salt thereof. Each possibility represents a separate embodiment. Currently preferred is the use of gentamicin or a pharmaceutically acceptable salt thereof (e.g. gentamicin sulfate).

Suitable glycopeptide antibiotics within the scope of the present invention include, but are not limited to, vancomycin, teicoplanin, telavancin, ramoplanin, decaplanin, corbomycin, complestatin, bleomycin, and a pharmaceutically acceptable salt thereof. Each possibility represents a separate embodiment. Currently preferred is the use of vancomycin or a pharmaceutically acceptable salt thereof (e.g. vancomycin hydrochloride).

Additional non-limiting antibiotics include a 5-nitroimidazole, a fluroquinolone, apramycin, arbekacin, astromicin, bekanamycin, dihydrostreptomycin, elsamitrucin, metronidazole, tinidazole, rifampicin, fosfomycin/tobramycin, G418, hygromycin B, isepamicin, kasugamycin, legonmycin, lividomycin, micronomicin, neamine, nourseothricin, paromomycin, plazomicin, ribostamycin, streptoduocin, totomycin, verdamicin, cephalothin, cefazolin, cephapririn, cephalexin, ciprofloxacin, levofloxacin, moxifloxacin, norfloxacin, azithromycin, clarithromycin, dirithromycin, erythromycin, and clindamycin, or a pharmaceutically acceptable salt thereof. Each possibility represents a separate embodiment.

It is to be understood that combinations of several antibiotics are contemplated by the present invention. For example, a composition comprising a combination of vancomycin with rifampicin, or pharmaceutically acceptable salts thereof; or a composition comprising a combination of minocycline with rifampicin, or pharmaceutically acceptable salts thereof are meant to be included within the scope of the present invention.

Typical amounts of the antibiotics to be incorporated into the composition of the present invention are from about 0.01% to about 20% (w/v) of the total weight of the composition, including each value within the specified range. Typical ranges include, but are not limited to, about 0.1% to about 15% (w/v), and about 0.5% to about 10% (w/v), including each value within the specified ranges. Exemplary amounts include, but are not limited to, about 0.01%, about 0.05%, about 0.1% about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19% or about 20% (w/v) of the total weight of the composition. Each possibility represents a separate embodiment.

In some embodiments, the composition affords the modified local release of said antibiotics. In one embodiment, the composition provides the sustained release of said antibiotics such that it is released over an extended period of time when administered to the tissue. For example, the antibiotics can be released over a period of about 3 hours to about 60 days, about 12 hours to about 30 days, about 24 hours to about 20 days, or about 2 to about 15 days, including each value within the specified ranges. Typical release periods include, but are not limited to, about 3 hours, about 6 hours, about 12 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days about 21 days, about 22 days, about 23 days, about 24 days, about 25 days, about 26 days, about 27 days, about 28 days, about 29 days, about 30 days, about 35 days, about 40 days, about 45 days, about 50 days, about 55 days, about 60 days or more, with each possibility representing a separate embodiment.

According to some embodiments, the pharmaceutical composition further comprises at least one pharmaceutically acceptable excipient. Suitable pharmaceutically acceptable excipients include, but are not limited to, at least one of a chelating agent, a buffering or pH adjusting agent, a preservative, an anti-oxidant, a thickening agent, a tonicity enhancing agent, a tissue adhesive, an inorganic mineral, and a combination or mixture thereof. Each possibility represents a separate embodiment.

Suitable chelating agents within the scope of the present invention include, but are not limited to, disodium edetate, deferoxamine mesylate (desferrioxamine), 2,3-dimercaprol, meso-2,3-dimercaptosuccinic acid (DMSA) and its ester analogues, deferiprone, nitrilotriacetic acid (NTA) and combinations thereof, with each possibility representing a separate embodiment. The amount of the chelating agent in the composition, if present, is in the range of from about 0.01% to about 5% (w/v) of the total weight of the composition, including each value within the specified range. Exemplary amounts include, but are not limited to, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or about 5% (w/v) of the total weight of the composition, with each possibility representing a separate embodiment.

Suitable buffering or pH adjusting agents within the scope of the present invention include, but are not limited to, acid and bases such as hydrochloric acid, aspartic acid, glutamic acid, sodium hydroxide, potassium hydroxide, arginine, lysine, and a mixture or combination thereof. Each possibility represents a separate embodiment. In some embodiments, the ratio between the basic components of the composition and the acid components of the composition is in the range of about 1:0.1 to about 1:6, for example about 1:0.1, about 1:0.2, about 1:0.3, about 1:0.4, about 1:0.5, about 1:0.6, about 1:0.7, about 1:0.8, about 1:0.9, about 1:1, about 1:1.1, about 1:1.2, about 1:1.3, about 1:1.4, about 1:1.5, about 1:1.6, about 1:1.7, about 1:1.8, about 1:1.9, about 1:2, about 1:2.1, about 1:2.2, about 1:2.3, about 1:2.4, about 1:2.5, about 1:2.6, about 1:2.7, about 1:2.8, about 1:2.9, about 1:3, about 1:3.1, about 1:3.2, about 1:3.3, about 1:3.4, about 1:3.5, about 1:3.6, about 1:3.7, about 1:3.8, about 1:3.9, about 1:4, about 1:4.1, about 1:4.2, about 1:4.3, about 1:4.4, about 1:4.5, about 1:4.6, about 1:4.7, about 1:4.8, about 1:4.9 about 1:5, about 1:5.1, about 1:5.2, about 1:5.3, about 1:5.4, about 1:5.5, about 1:5.6, about 1:5.7, about 1:5.8, about 1:5.9, or about 1:6, with each possibility representing a separate embodiment. It is contemplated that the ratio between basic and acid components of the composition is calculated to incorporate all components present in the composition including the peptide and the proton donor(s) or acceptor(s), as well as the thickening agent(s), active pharmaceutical ingredient(s), tissue adhesive(s), and inorganic mineral(s) that may be present in the composition.

According to the principles of the present invention one or more buffering agents may be included in the composition. Such buffering agents include, but are not limited to, 2-amino-2-hydroxymethyl-1,3-propanediol (Tris), 2-[bis(2-hydroxyethyl)imino]-2-(hydroxymethyl)-1,3-propanediol (bis-Tris), 4-morpholine ethane sulfonic acid (MES) buffer, ammonium chloride, bicine, tricine, sodium phosphate monobasic, sodium phosphate dibasic, sodium carbonate, sodium bicarbonate, sodium acetate, sodium phosphate, glutamic acid, citrate buffer, Dulbecco's phosphate-buffered saline, 4-(2-hydroxyethyl) piperazineethanesulfonic acid (HEPES), methoxypsoralen (MOPS), N-cyclohexyl aminopropanesulfonic acid (CAPS), N-cyclohexyl-2-hydroxyl-3-aminopropanesulfonic acid (CAPSO), N-Cyclohexyl-2-aminoethanesulfonic acid (CHES), 3-[4-(2-Hydroxyethyl) piperazinyl]propanesulfonic acid (HEPPS), phosphate-buffered saline, tris-buffered saline, Hank's solution, and Ringer's solution. Each possibility represents a separate embodiment.

According to various aspects and embodiments, the composition has a pH in the range of about 5 to about 7, including each value within the specified range. Exemplary non-limiting ranges include about 5.3 to about 6.3, about 5.8 to about 6.8, about 6.0 to about 6.6 etc., including each value within the specified ranges. For example, the composition of the present invention may have a pH of about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, or about 7, with each possibility representing a separate embodiment.

Suitable preservatives within the scope of the present invention include, but are not limited to, parabens such as methylparaben, methylparaben sodium, ethylparaben, propylparaben, propylparaben sodium, butylparaben, and the like; cresols such as cresol, chlorocresol, and the like; phenolmercuric chloride, phenolmercuric acetate, acetomeroctol, nitromersol, thimerosal, mercurochrome, mercuric chloride, and mercuric iodide; elemental metals, such as silver and copper; and metal compounds, such as copper chloride, copper sulfate, copper peptides, zinc chloride, zinc sulfate, silver nitrate, silver iodide, silver acetate, silver benzoate, silver carbonate, silver chloride, silver citrate, silver oxide, silver sulfate and tincture of iodine. Each possibility represents a separate embodiment. The preservative can also be selected from other known agents including, but not limited to, hydroquinone, pyrocatechol, resorcinol, 4-n-hexyl resorcinol, benzalkonium chloride, benzalkonium chloride solution, benzethonium chloride, benzoic acid, benzyl alcohol, cetylpyridinium chloride, chlorobutanol, dehydro acetic acid, o-phenylphenol, phenol, phenyl ethyl alcohol, potassium benzoate, potassium sorbate, sodium benzoate, sodium dehydroacetate, sodium propionate, sorbic acid, thimerosal, thymol, phenylmercuric compounds such as phenylmercuric borate, phenylmercuric nitrate and phenylmercuric acetate, formaldehyde, and formaldehyde generators. Each possibility represents a separate embodiment.

Suitable antioxidants within the scope of the present invention include, but are not limited to, ascorbic acid, N-acetyl cysteine (NAC), derivatives and salts thereof. N-acetylcysteine (NAC) is used as a food supplement aimed to supply cells with cysteine thereby increasing glutathione (GSH) cellular levels. It is known to act as a precursor for the synthesis of GSH, which is a very efficient redox scavenger, carrying a free thiol group that can interact directly with reactive oxygen/nitrogen species and maintain the oxidative status of key cellular enzymes. NAC has been used as an anti-oxidant, for example to treat paracetamol (acetaminophen) overdose. It has also been known to exert beneficial therapeutic effect in loosening the thick mucus in individuals with cystic fibrosis or chronic obstructive pulmonary disease (COPD). NAC has further been suggested to exert antimicrobial properties and reduce biofilm formation in a variety of gram-positive and gram-negative bacteria, including Pseudomonas aeruginosa, Klebsiella pneumoniae, Staphylococcus epidermidis, Staphylococcus aureus, and Escherichia coli.

In some aspects and embodiments, the composition of the present invention comprises NAC or derivatives thereof affording their endogenous antimicrobial properties in the absence of antibiotics. In other aspects and embodiments, the composition of the present invention comprises NAC or derivatives thereof in combination with an antibiotic as disclosed herein.

NAC and derivatives thereof within the scope of the present invention include, but are not limited to, N-acetylcysteine, S-(2-(1-carboxy-2-methylpropyl)isoindole-1-yl)-N-acetylcysteine, N-acetylcysteine lysinate, S-phenyl-N-acetylcysteine, N-acetyl-S-(N-methylcarbamoyl)cysteine, N-acetyl-S-pentachloro-1,3-butadienylcysteine, adamantyl-N-acetylcystein, N-acetylcysteinamide, S-(1-(4′-methoxyphenyl)-2-hydroxypropyl)-N-acetylcysteine, S-(N,N-diethyldithiocarbamoyl)-N-acetylcysteine, S-(6-purinyl)-N-acetylcysteine, S-(3-oxopropyl)-N-acetylcysteine, S-(2-carboxyethyl)-N-acetylcysteine, S-(3-hydroxy-3-carboxy-n-propyl)-N-acetylcysteine, N-acetylcysteine-6,7-dihydro-7-hydroxy-1-hydroxymethyl-5H-pyrrolizine, S-(2-(N(7)-guanyl)ethyl)-N-acetylcysteine, S-trichlorovinyl-N-acetylcysteine, S-(2-methylbenzyl)-N-acetylcysteine, S-1,2-dichlorovinyl-N-acetylcysteine, S-(2-((1-methylethyl)phenylamino)-2-oxoethyl)-L-cysteine, and S-nitroso-N-acetylcysteine. Each possibility represents a separate embodiment. Typically, when antioxidant is added to the composition, it is in an amount of from about 0.01% to about 20% (w/v) of the total weight of the composition, including each value within the specified range. Exemplary ranges of the amount of anti-oxidant in the composition include, but are not limited to, about 0.05% to about 15%, about 0.1% to about 10%, about 0.1% to about 5%, or about 0.2% to about 4% (w/v) of the total weight of the composition, including each value within the specified ranges.

Suitable thickening agents within the scope of the present invention include, but are not limited to, hydroxypropyl methylcellulose, hydroxypropyl cellulose, hydroxyethyl cellulose, hydroxymethyl cellulose, carboxy methyl cellulose, polyvinylpyrrolidone, polyvinyl alcohol, and salts thereof. Typically, the amount of the thickening agent in the composition, if present, is in the range of from about 0.1% to about 10% (w/v) of the total weight of the composition including each value within the specified range. Exemplary ranges include, but are not limited to about 0.5% to about 5%, about 1% to about 4%, or about 1.5% to about 3% (w/v) of the total weight of the composition, including each value within the specified ranges.

Suitable tonicity enhancing agents within the scope of the present invention include, but are not limited to, polyol such as mannitol, glycerol, sorbitol, xylitol and combinations thereof, with each possibility representing a separate embodiment. Typically, the amount of the tonicity enhancing agent in the composition, if present, is in the range of from about 0.1% to about 30% (w/v) of the total weight of the composition, including each value within the specified range. Exemplary amounts include, but are not limited to, about 0.2% to about 20%, about 1% to about 10%, or about 2% to about 7% (w/v) of the total weight of the composition, including each value within the specified ranges.

Suitable tissue adhesives that may be incorporated in the compositions of the present invention include, but are not limited to, fibrin(ogen), gelatin, collagen, chitosan, cyanoacrylate, and combinations thereof, with each possibility representing a separate embodiment. Typically, the amount of the tissue adhesive in the composition, if present, is in the range of from about 1% to about 20% (w/v) of the total weight of the composition, including each value within the specified range. Exemplary ranges include, but are not limited to, about 1% to about 15%, about 5% to about 15%, or about 8% to about 12% (w/v) of the total weight of the composition, including each value within the specified ranges.

Suitable inorganic minerals that may be incorporated in the compositions of the present invention include, but are not limited to, hydroxyapatite, calcium phosphate, calcium carbonate, calcium gluconate, calcium oxalate, calcium sulfate, calcium chloride, magnesium phosphate, magnesium carbonate, magnesium gluconate, magnesium oxalate, magnesium sulfate, magnesium chloride, zinc phosphate, zinc carbonate, zinc gluconate, zinc oxalate, zinc sulfate, zinc chloride, sodium bicarbonate, and a mixture or combination thereof. Each possibility represents a separate embodiment. In currently preferred embodiments, the inorganic mineral is a calcium phosphate mineral selected from the group consisting of amorphous calcium phosphate, tricalcium phosphate, β-tri-calcium phosphate, and hydroxyapatite. Each possibility represents a separate embodiment. Typically, the amount of the inorganic mineral in the composition, if present, is in the range of from about 5% to about 95% (w/v) of the total weight of the composition, including each value within the specified range. Exemplary amounts include, but are not limited to, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% (w/v) of the total weight of the composition, with each possibility representing a separate embodiment.

The compositions of the present invention may be manufactured by processes well known in the art, e.g., by means of conventional mixing, dissolving, suspending, solubilizing, complexing, granulating, levigating, emulsifying, encapsulating, entrapping, spray-drying, and lyophilizing processes, or a combination thereof. They may be formulated in a conventional manner using one or more pharmaceutically acceptable excipients as described above, which facilitate processing of the peptides and peptide derivatives and salts into preparations which can be used as medicaments. For example, the peptide or derivative or salt thereof is typically admixed with a thickening agent with or without inorganic mineral(s). The antibiotics and/or NAC, derivatives or salts thereof are typically dissolved or suspended in an aqueous phase optionally comprising chelating agent(s), buffering or pH adjusting agent(s), tonicity enhancing agent(s), tissue adhesive(s), and/or preservative(s). The aqueous phase is then typically combined with the peptide and processed so as to form the composition of the present invention. Additional embodiments include the formation of a hydrogel by mixing the aqueous phase comprising the antibiotics and/or NAC, derivatives or salts thereof with the peptide, derivative or salt thereof and other excipients as described above.

Further embodiments include the formation of a hydrogel by mixing an aqueous phase with the peptide derivative or salt thereof and the antibiotics and/or NAC, derivatives or salts thereof. Following the formation of a stable hydrogel, additional excipients as described above can be added to yield e.g. a putty.

According to certain aspects and embodiments, the composition disclosed herein further comprises an antiresorptive agent and/or an anti-inflammatory agent. Each possibility represents a separate embodiment.

When an antiresorptive agent and/or an anti-inflammatory agent is incorporated into the composition of the present invention, its concentration is typically in the range of about 0.01 to about 200 mg/g of the total weight of the composition, including each value within the specified range. Exemplary ranges include, but are not limited to, about 0.1 to about 200 mg/g, about 0.5 to about 150 mg/g, about 1 to about 100 mg/g, about 5 to about 50 mg/g, or about 10 to about 20 mg/g, including each value within the specified ranges. Typical concentrations include, but are not limited to, about 0.01, about 0.05, about 0.1, about 0.5, about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, or about 200 mg/g, with each possibility representing a separate embodiment.

Suitable antiresorptive agents within the scope of the present invention include, but are not limited to, a bisphosphonate selected from the group consisting of zoledronic acid, pamidronate, alendronate, etidronate, clodronate, risedronate, tiludronate, ibandronate, incadronate, minodronate, olpadronate, neridronate, and EB-1053, or a pharmaceutically acceptable salt thereof. Each possibility represents a separate embodiment.

Suitable anti-inflammatory agents within the scope of the present invention include, but are not limited to, a non-steroidal anti-inflammatory drug (NSAID). Exemplary non-limiting NSAIDs include ibuprofen, flurbiprofen, diclofenac, and naproxen, or a pharmaceutically acceptable salt thereof. Each possibility represents a separate embodiment.

In other aspects and embodiments, a chemotherapeutic drug is further incorporated into the composition. Suitable chemotherapeutic drugs include, but are not limited to, doxorubicin, cyclophosphamide, fluorouracil (5-fluorouracil or 5-FU), methotrexate, bleomycin, thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb and bevacizumab, or a pharmaceutically acceptable salt thereof. Each possibility represents a separate embodiment.

Additional therapeutic agents that may be incorporated into the composition of the present invention include, but are not limited to, antiseptic agents such as chlorhexidine and salts thereof, bioactive agents such as cytokines, growth factors and their activators etc. that enhance the bone repair and regeneration. Representative proteins include, but are not limited to, bone growth factors (bone morphogenetic proteins or insulin-like growth factors) and fibroblast growth factors as well as retinoids, growth hormone (GH), leptin and transferrin. Each possibility represents a separate embodiment. Additionally, cells genetically engineered to express the aforementioned proteins are included within the scope of the present invention and may also be incorporated into the compositions disclosed herein. Preferred examples for bone repair and regeneration uses include progenitor, periosteal or other mesenchymal stem cells or specialized cells such as, but not limited to, chondrocytes, osteocytes, and/or osteoblasts per se or cells transfected with bone growth factor genes. Other bioactive agents that may be incorporated in the compositions of the present invention include blood factors that regulate clot formation such as fibrin and plasminogen.

In certain aspects and embodiments, the composition of the present invention may be in a liquid, semi-solid or solid preparation form such as, but not limited to, an emulsion, a hydrogel or a putty. Each possibility represents a separate embodiment. The term “hydrogel” according to the present invention refers to a three-dimensional hydrated assembly of bioactive nanofibers. This definition includes dry “hydrogel forming peptides” that will swell in aqueous environments, as well as water-swollen materials. Typically, in hydrated hydrogels, according to the principles of the present invention, the amount of water in the composition ranges from about 1% to about 99% (w/v) of the total weight of the composition, including each value within the specified range. Exemplary ranges of water content include, but are not limited to, about 10% to about 20%, about 30% to about 40%, about 50% to about 70%, about 75% to about 95%, about 80% to about 99% (w/v) of the total weight of the composition, including each value within the specified ranges. A hydrogel according to the present invention can be tailored to possess a range of properties depending on the peptides of which the hydrogel is composed and on additional materials that may be added thereto. The composition, preferably in the form of a stable hydrogel, may be used as a medicament per se for treatment in the operating room or clinics. Alternatively, it can be provided as a kit to be reconstituted in situ.

Methods of Use

In some aspects and embodiments, the compositions of the present invention are useful in treating or preventing various diseases or disorders, particularly bacterial infections, which are associated with mineralized tissues. In accordance with these embodiments, there is provided a method of treating or preventing a disease or disorder associated with mineralized tissue, the method comprising administering to a subject in need thereof a therapeutically effective amount of the composition of the present invention. As used herein, the term “treating” refers to abrogating, inhibiting, slowing, reversing, or preventing the progression of a disease, ameliorating clinical symptoms of a disease or preventing the appearance of clinical symptoms of a disease. The term “administering” as used herein refers to bringing into contact with the composition of the present invention thereby providing the aforementioned therapeutic benefits to a subject, preferably a human subject. The tissue to which the composition may be administered includes, but is not limited to, a hard tissue or a soft tissue, with each possibility representing a separate embodiment. The term “hard tissue” as used herein refers to a tissue that has become mineralized, such as, for example, bone, cartilage, and tooth and the term “soft tissue” as used herein refers to a non-mineralized connective tissue. It is contemplated that the soft tissue to which the composition is administered is near or in proximity to the hard tissue thereby affording local treatment of diseases or disorders which are associated with the hard tissue.

Diseases or disorders within the scope of the present invention include, but are not limited to, subchondral bone lesions, osteomyelitis, peri-prosthetic joint infections, and surgery site infections. Each possibility represents a separate embodiment. Additional diseases or disorders include, but are not limited to, infections in subject having primary joint replacement, revision joint replacement, avascular necrosis, high risk contralateral hip fractures, delayed union fractures, non-union fractures, open fractures, cartilage transplant, and stress fractures. Each possibility represents a separate embodiment. Further included within the scope of the present invention are jaw osteonecrosis and peri-mucositis. Each possibility represents a separate embodiment.

According to certain aspects and embodiments, there is provided a method of treating an infected tissue in a subject in need thereof, the method comprising the step of locally administering to said tissue a composition as disclosed herein.

According to other aspects and embodiments, there is provided a method of treating a periodontal disease selected from periodontitis, periimplantitis, and jaw osteonecrosis, the method comprising the step of administering to a subject in need thereof a composition as disclosed herein.

According to various aspects and embodiments, there is provided a method of treating bone related infections selected from subchondral bone lesions, osteomyelitis, and peri-prosthetic joint infection and preventing a surgery site infection (SSI), the method comprising the step of administering to a subject in need thereof a composition as disclosed herein. In one embodiment, the bone related infection is diabetic foot ulcer.

According to certain aspects and embodiments, the compositions of the present invention are useful as coatings to implants prior to their implantation. The implants can be metal implants, metal oxide implants and/or ceramic implants. Each possibility represents a separate embodiment.

According to other aspects and embodiments, there is provided a method of treating or preventing an implant-related infection, the method comprising the step of administering to a subject in need thereof a therapeutically effective amount of a composition as disclosed herein.

According to further aspects and embodiments, there is provided a method of treating or preventing an infection selected from eye infection, urinary infection, vaginal infection, ovarian infection, and rectal infection, the method comprising the step of administering to a subject in need thereof a therapeutically effective amount of a composition as disclosed herein.

Treatment of an infected tissue is meant to cover the inhibition of bacteria replication with reduction of bacterial load or even complete eradication of the bacteria upon treatment. In specific embodiments, the composition of the present invention is useful in treating periodontitis. In other embodiments, the composition of the present invention is useful in treating periimplantitis. In further embodiments, the composition of the present invention is useful in treating peri-mucositis. In treating periodontitis, periimplantitis, or peri-mucositis the administration is typically affected to a soft tissue that is near or in proximity to a hard tissue such as, but not limited to, a periodontal tissue, for example into a periodontal pocket near a tooth or a dental implant. According to the principles of the present invention, the compositions disclosed herein were surprisingly found to be effective in the treatment of new patient populations, such as in patients with progressive periodontal diseases. These patients include subjects afflicted with periodontitis and/or periimplantitis. Each possibility represents a separate embodiment. Within the scope of the present invention are subjects having periodontal implants thereby being susceptible for developing peri-implantitis. Additional patient population is of patients that have already developed periodontitis and/or peri-implantitis and are not suitable for or are resistant to conventional nonsurgical treatments such as mechanical debridement, and administration of antiseptics and/or antibiotics. For example, the compositions disclosed herein can be used to treat patients having periodontitis and/or peri-implantitis for which conventional therapeutic modalities are inadequate or insufficient. The invention advantageously provides for the treatment of these new patient populations with enhanced efficacy and/or safety while minimizing adverse side effects.

In accordance with these embodiments, there is provided a method of treating a progressive periodontal disease selected from periodontitis and peri-implantitis in a subject in need thereof, the method comprising the step of locally administering to a periodontal tissue in proximity to a tooth or a dental implant a composition as disclosed herein.

In one embodiment, treatment of peri-implantitis comprises prophylactic treatment to subjects having dental implants but are not yet afflicted with peri-implantitis. In accordance with these embodiments, the composition of the present invention may be administered together with the insertion of the dental implant or shortly thereafter to prevent the formation of peri-implantitis. In one embodiment, the composition of the present invention may be administered to subjects afflicted with gingivitis or peri-implant mucositis in order to prevent their deterioration to periodontitis or periimplantitis, respectively.

In some embodiments, the pharmaceutical composition of the present invention is useful in reducing or suppressing bone loss supporting a tooth or a peri-implant in subjects having longitudinal overt progressive bone loss of 3 mm or more. Treatment therefore comprises at least about 5% to at least about 90% reduction in bone loss, including each value within the specified range. For example, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or even about 100% reduction in bone loss is contemplated by the present invention, with each possibility representing a separate embodiment. In accordance with these embodiments, a reduction in bone loss comprises a longitudinal overt bone loss in the range of about 2.85 to about 0.3 mm, including each value within the specified range. For example, the longitudinal overt bone loss is about 2.85, about 2.8, about 2.75, about 2.7, about 2.65, about 2.6, about 2.55, about 2.5, about 2.45, about 2.4, about 2.35, about 2.3, about 2.25, about 2.2, about 2.15, about 2.1, about 2.05, about 2.0, about 1.95, about 1.9, about 1.85, about 1.8, about 1.75, about 1.7, about 1.65, about 1.6, about 1.55, about 1.5, about 1.45, about 1.4, about 1.35, about 1.3, about 1.25, about 1.2, about 1.15, about 1.1, about 1.05, about 1.0, about 0.95, about 0.9, about 0.85, about 0.8, about 0.75, about 0.7, about 0.65, about 0.6, about 0.55, about 0.5, about 0.45, about 0.4, about 0.35, or about 0.3 mm, with each possibility representing a separate embodiment.

In other embodiments, the pharmaceutical composition of the present invention is useful for inducing bone repair and regeneration of the hard tissue supporting a tooth or a peri-implant. Without being bound by any theory or mechanism of action, the bone repair and regeneration is afforded by inducing bone formation which may be enhanced either by recruiting osteoblasts, the bone forming cells, or by inhibiting recruitment or activity of osteoclasts, the bone resorbing cells. It is contemplated that the composition of the present invention is suitable for supporting and facilitating cellular growth. Accordingly, various types of cells can be incorporated into the composition including, but not limited to, stem cells or progenitor cells or specialized cells such as, but not limited to, osteoblasts. The peptides of the present invention are particularly useful in forming a matrix to support biomineralization thereby inducing the formation of regenerated bone around the tooth or the peri-implant.

In other embodiments, the pharmaceutical composition of the present invention is useful in reducing the depth of a periodontal pocket around a tooth or a peri-implant in subjects having a Periodontal Pocket Depth (PPD) of 5 mm or more. Treatment therefore comprises at least about 5% to at least about 90% reduction in PPD, including each value within the specified range. For example, at least about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% reduction in PPD is contemplated by the present invention, with each possibility representing a separate embodiment. In accordance with these embodiments, a reduction in PPD comprises PPD in the range of about 4.75 to about 0.5 mm, about 4 to about 1 mm, or about 3.5 to about 1.5 mm, including each value within the specified ranges. For example, the PPD following treatment is about 4.75, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, or about 0.5 mm, with each possibility representing a separate embodiment.

It is to be understood that treatment, according to the principles of the present invention, may further be accompanied with other treatment modalities including, for example, mechanical debridement.

In additional embodiments, the composition of the present invention is useful in treating jaw osteonecrosis. In other embodiments, the composition of the present invention is useful in treating inflammatory diseases such as bone marrow lesions and osteoarthritis.

According to further aspects and embodiments, the composition of the present invention is useful in repairing and regenerating bone and/or promoting angiogenesis. In accordance with these embodiments, the composition is useful in repairing bone defects and enhancing bone substitution and healing in conditions such as, but not limited to, osteopenia, osteoporosis, etc., and promoting orthopedic regeneration and healing in conditions such as primary joint replacement, revision joint replacement, cartilage wear, avascular necrosis etc.

According to the principles of the present invention there is provided a method of treating inflammation including bone marrow lesions and osteoarthritis, the method comprising administering to a subject in need thereof a composition as disclosed herein.

In additional aspects and embodiments, the composition of the present invention is also useful in treating cancer in a subject in need thereof when incorporating a chemotherapeutic agent. Site-specific chemotherapy that provides high drug concentrations for an extended time period in the diseased site is an effective way of treating remnant infected cells after resection of the infected area such as solid tumors. Furthermore, the efficacy of systemic chemotherapeutic drug treatments is impeded when the tumor resides in a tissue such as bone tissue due to lack of ability of the drug to penetrate the exact location in the bone thereby targeting the specific cancerous cells. The composition of the present invention provides targeted delivery of the chemotherapeutic drug to afford local and/or systemic therapy. In some embodiments, a composition comprising a chemotherapeutic drug is useful in treating a primary bone cancer such as, but not limited to, osteosarcoma, Ewing sarcoma, chondrosarcoma, and chordoma; bone metastases and cysts; giant cell tumor; and multiple myeloma related bone lesions. Each possibility represents a separate embodiment.

Further included within the scope of the present invention is the treatment of various eye-related diseases or disorders including, but not limited to, dry eye syndrome, glaucoma, fuchs' corneal dystrophy, cataract or trauma induced following cataract surgery, corneal erosion, and age-related macular degeneration. Each possibility represents a separate embodiment.

The compositions of the present invention can be formulated to suit any route of administration chosen. In particular, the pharmaceutical compositions of the present invention are formulated for topical, transdermal, intra-lesion, intra-uterine, intra-bladder, intra-ocular, intra-auricular, intra-articular, intra-body cavities, intra-muscular, intra-cutaneous, intra-osseous, subchondral, and subcutaneous administration. Each possibility represents a separate embodiment. When treatment or prevention of a disease or disorder related to an implant is desired, the composition can be spread or injected into a crevice or opening near a mineralized tissue. Spreading or injection of the composition may be performed together with the insertion of the implant or after its insertion. Alternatively, the implant may be coated with the composition of the present invention.

The effective therapeutic amount to be administered is typically determined by one of ordinary skill in the art. Factors to consider in determining a therapeutically effective amount include age, weight and physical condition of the person to be treated, type of agent used, and desired release rate. Typically, the compositions are administered by injection using a suitable syringe and/or implantation into cavities or any organ or tissue within a subject in need thereof to afford local and/or systemic therapy. The compositions may also be spread on the surface of different body tissues and/or mucosa and may be administered in adjunct to a surgical procedure. Additional embodiments include the use of the compositions disclosed herein as coatings on implantable medical devices, such as stents, catheters, implants, and surgical sealants.

As used herein the term “about” refers to ±10%.

The terms “comprise”, “comprising”, “include”, “including”, “having” and their conjugates mean “including, but not limited to”.

As used herein, the singular form “a”, “an” and “the” include plural references unless the context clearly dictates otherwise. For example, the term “a peptide” or “an excipient” may include a plurality of peptides and excipients, including mixtures thereof.

Throughout this application, various embodiments of the invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number to a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions, illustrate the invention in a non-limiting fashion.

### Example 1. Stable Hydrogel Formation without Tetracycline Antibiotics

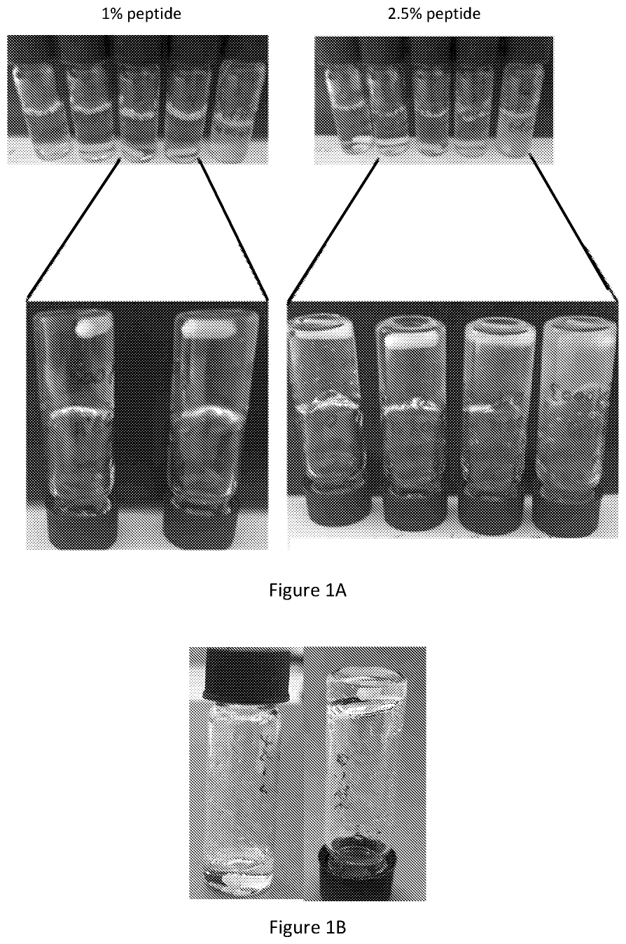

Different mole ratios of PFD5 sodium to HCl were generated by dissolving the peptide in an HCl solution using a magnetic stirrer. Following mixing for a few minutes, hydrogel formation was tested (Table 2 and FIG. 1A-1B). Compositions containing 1% (w/v) of PFD5 at a concentration of 6 mM (equivalent to 36 mM COO⁻), resulted in stable hydrogels with the addition of 18 mM HCl. Hence, in order to obtain a clear and homogeneous hydrogel, the mole ratio of the peptide to acid was determined to be at least 1:3. Without being bound by any theory or mechanism of action, it is assumed that when the peptide is at its pKₐ (pH˜5.95) thereby exhibiting half of the Asp residues protonated, a stable hydrogel is formed. Increasing the peptide to acid ratio to 1:4 resulted in a stable hydrogel that is opaque. At a 1:6 ratio, the stable hydrogel appeared opaquer.

Compositions containing 2.5% (w/v) of PFD5 at a concentration of 15 mM (equivalent to 90 mM COO⁻) resulted in stable hydrogels with the addition of 30 mM HCl. Hence, in order to obtain a clear and homogeneous hydrogel at this peptide concentration, the mole ratio of the peptide to acid was determined to be 1:2. Without being bound by any theory or mechanism of action, it is assumed that at this pH (pH=6.37) the peptide exhibits a third of the Asp residues protonated, resulting in a stable hydrogel. At a 1:4 ratio, the stable hydrogel appeared opaquer.

Compositions containing 4.8% (w/v) of PFD5 at a concentration of 28.8 mM (equivalent to 173 mM COO⁻) resulted in stable hydrogels with the addition of 48 mM HCl. At this peptide concentration, the mole ratio of the peptide to acid was determined to be 1:1.67 to afford a stable clear hydrogel (FIG. 1B).

TABLE 2

| | | | | | Molar Ratio | | Gel | |
|---|---|---|---|---|---|---|---|---|
| | Peptide | peptide | COO⁻ | HCl | peptide | | assessment | |
| Formulation | % w/v | mM | mM | mM | (base):acid | pH | HD | Clarity |
| #1 | 1 | 6 | 36 | 1 | 1:0.17 | 6.68 | N | Y |
| #2 | 1 | 6 | 36 | 4 | 1:0.67 | 6.08 | N | Y |
| #3 | 1 | 6 | 36 | 12 | 1:2 | 5.98 | N | Y |
| #4 | 1 | 6 | 36 | 18 | 1:3 | 5.95 | Y | Y |
| #5 | 1 | 6 | 36 | 24 | 1:4 | 5.44 | Y | O |
| #6 | 1 | 6 | 36 | 36 | 1:6 | 4.11 | N | O |
| #7 | 2.5 | 15 | 90 | 2.5 | 1:0.15 | 6.61 | N | Y |
| #8 | 2.5 | 15 | 90 | 10 | 1:0.67 | 6.39 | N | Y |
| #9 | 2.5 | 15 | 90 | 30 | 1:2 | 6.37 | Y | Y |
| #10 | 2.5 | 15 | 90 | 45 | 1:3 | 5.99 | Y | O |
| #11 | 2.5 | 15 | 90 | 60 | 1:4 | 5.46 | Y | O |
| #12 | 2.5 | 15 | 90 | 90 | 1:6 | 3.95 | Y | O |
| #12a | 3.75 | 22.5 | 135 | 45 | 1:2 | 6.33 | Y | Y |
| #12b | 4.8 | 29 | 174 | 48 | 1:1.6 | 6.7 | Y | Y |

HD: Hydrogel formation; Y = Yes; N = No; O = Opaque.

### Example 2. Hydrogel and Putty Formation with Tetracycline Antibiotics

Doxycycline hyclate was dissolved in deionized water to form a clear solution at a concentration of 5% w/v (91 mM doxycycline and 91 mM HCl; pH=1.93). At this pH, doxycycline is protonated. A composition containing 1.7 ml PFD5 hydrogel (2.5% dissolved in 30 mM HCl, formulation #9) was mixed with 0.5 ml of the doxycycline hyclate solution. At pH=4.4 no gel was formed. Rather, an opaque solution was obtained (formulation #13).

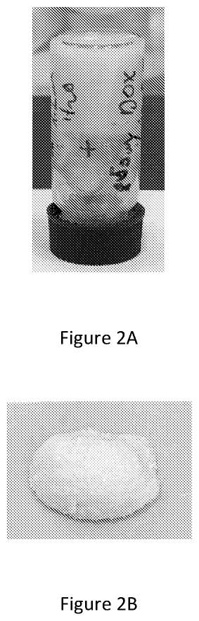

A hydrogel at pH=5.8 (formulation #13-1) was prepared by adding to the above formulation (#13), 60 μl NaOH 1M. The hydrogel was complemented with 1,300 mg of tricalcium phosphate (TCP) to form a putty. Another hydrogel at pH=6.06 (FIG. 2A, formulation #14) was prepared and supplemented with 1,040 mg of TCP mixed with 20 mg carboxy methyl cellulose (CMC) to form a putty (FIG. 2B).

Additional hydrogels with different peptide to doxycycline ratios were prepared as detailed in Table 3.

TABLE 3

| | | | Doxy- | | | | | Doxy- | | | | | | |
|---|---|---|---|---|---|---|---|---|---|---|---|---|---|---|
| | Peptide | | cycline | | Extra | | | cycline | Doxy- | Total | | Peptide: | | Gel |
| | mg & % | Diluent | mg & % | Diluent | HCl 1M | Final | Peptide | mM + | cycline | HCl | Peptide: | Doxy- | | assess- |
| # | w/v | ml | w/v | ml | in ml | Volume | mM & % | HCl | % w/v | mM | HCl | cycline | pH | ment |
| #14 | 5012.5 | 0.4 | 1006.2 | 1.6 | | 2 | 152.5 | 91.7 | 5 | 91.7 | 1:6 | 1:6 | 2.9 | N |
| | | | | | | | | | | | | | | O |
| #15 | 5012.5 | 0.4 | 503.1 | 1.6 | | 2 | 152.5 | 45.8 | 2.5 | 45.8 | 1:3 | 1:3 | 6 | Y |
| | | | | | | | | | | | | | | O |
| #16 | 5010 | 0.5 | 100.67 | 1.5 | 0.024 | 2 | 152.5 | 9.1 | 0.5 | 21.1 | 1:1.4 | 1:0.6 | 6.4 | Y* |
| | | | | | | | | | | | | | | W |
| #17 | 5010 | 0.5 | 201.3 | 1.5 | 0.01 | 2 | 152.5 | 18.2 | 1 | 23.2 | 1:1.5 | 1:1.2 | 6.5 | N |
| | | | | | | | | | | | | | | O |
| #18 | 5010 | 0.5 | 200.8 | 1.475 | 0.025 | 2 | 152.5 | 18.2 | 1 | 30.7 | 1:2 | 1:1.2 | 6.0 | Y** |
| | | | | | | | | | | | | | | O |
| | | | | | | | | | | | | | | W |
| #19 | 10010 | 1 | 202.1 | 0.951 | 0.049 | 2 | 305 | 18.2 | 1 | 42.7 | 1:1.4 | 1:0.6 | 6.7 | Y |
| | | | | | | | | | | | | | | C |
| | | | | | | | | | | | | | | S |
| #20 | 10010 | 1 | 202 | 1 | 0.05 | 2 | 305 | 18.2 | 1 | 43.2 | 1:1.4 | 1:0.6 | 6.5 | Y |
| | | | | | | | | | | | | | | C |
| | | | | | | | | | | | | | | W |
| #20a | 505 | 1 | 202 | 1 | 0.02 | 2 | 152.5 | 18.2 | 1 | 28.2 | 1:1.9 | 1:1.2 | 6.3 | Y |
| | | | | | | | | | | | | | | C |
| | | | | | | | | | | | | | | S |

Y = Yes;
N = No;
O = Opaque;
C = Clear;
W = weak;
S = strong
*formed after 40 minutes
**formed after 12 minutes

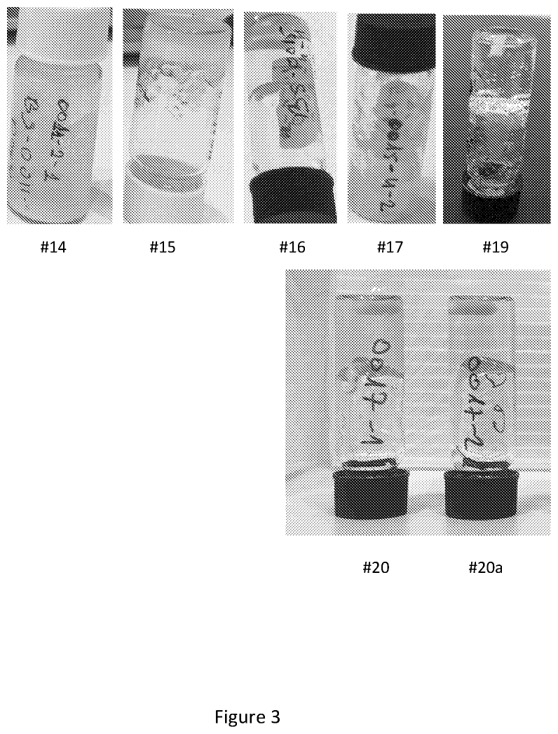

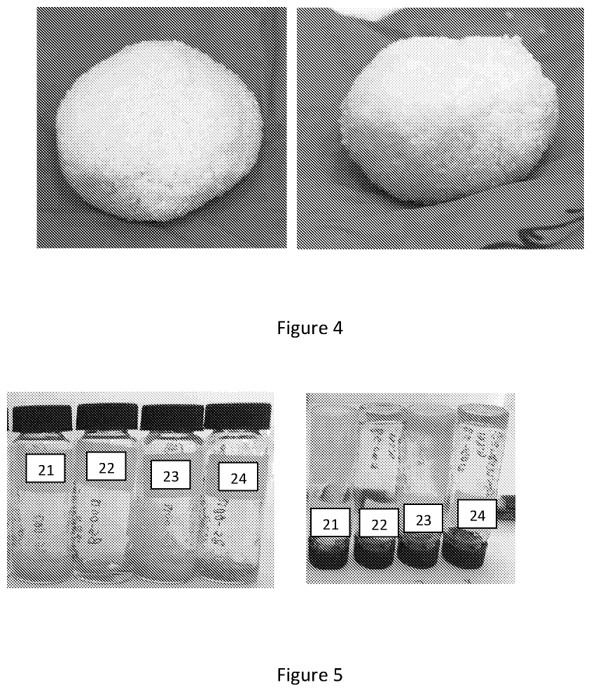

Images of the selected hydrogels are shown in FIG. 3. FIG. 4 shows images of a putty formed by supplementing 30 mg carboxy methyl cellulose (CMC) and 2,300 mg tricalcium phosphate (TCP) to formulation #17 yielding hand-malleable putty.

Hydrogel and putties with minocycline hydrochloride have been made (Table 4 and FIG. 5). Minocycline hydrochloride or a minocycline composition for injection containing 2% w/v minocycline (Minocin® 100 Mg/Vial each vial containing 100 mg minocycline hydrochloride (40 mM), and 269 mg magnesium sulfate heptahydrate (218 mM), dissolved in 5 ml water and adjusted with HCl to a pH of 4.5-5), PFD5 sodium, and HCl were added to a glycerin/CMC that was first dissolved in 5 ml of acidified water. The compositions were then lyophilized and reconstituted to a final volume of 1 ml. Resultant formulations were tested for hydrogel formation by the flipped vial quick assay.

TABLE 4

| | | | | CMC: | | | | Mino- | | | | | | |
|---|---|---|---|---|---|---|---|---|---|---|---|---|---|---|
| | | Mino- | Extra | glycerin | | | | cycline | Mino- | Total | | Peptide: | | Gel |
| | Peptide | cycline | HCl 1M | 15/70 mg/ | Final | Peptide | Peptide | mM + | cycline | HCl | Peptide: | Mino- | | assess- |
| # | mg | HCl mg | in μl | μL in mg | Volume | mM | w/v % | HCl | w/v % | mM | HCl | cycline | pH | ment |
| #21 | 25 | 10 | 10 | 85 | 1 | 15 | 2.5 | 20 | ~1 | 30 | 1:2 | 1:1.3 | 6.32 | N |
| #22 | 25 | 10 | 10 | — | 1 | 15 | 2.5 | 20 | ~1 | 30 | 1:2 | 1:1.3 | 6.31 | Y |
| #23 | 25 | 20 | 40 | 85 | 1 | 15 | 2.5 | 40 | ~2 | 80 | 1:4 | 1:2.7 | 6.13 | P |
| #24 | 25 | 20 | 40 | — | 1 | 15 | 2.5 | 40 | ~2 | 80 | 1:4 | 1:2.7 | 6.07 | Y |
| #24a | 37.5 | 20 | 20 | — | 1 | 22.5 | 3.75 | 40 | ~2 | 60 | 1:3 | 1:1.7 | 6.17 | Y |

Y = Yes;
N = No;
P = Partial

Formulations #23 and #24 were supplemented with 180 mg/ml TCP to yield putties that were tested by hand malleability to be slightly overhydrated and friable. Nonetheless, these results demonstrate that putties can be formed from an opaque solution (emulsion-like solution).

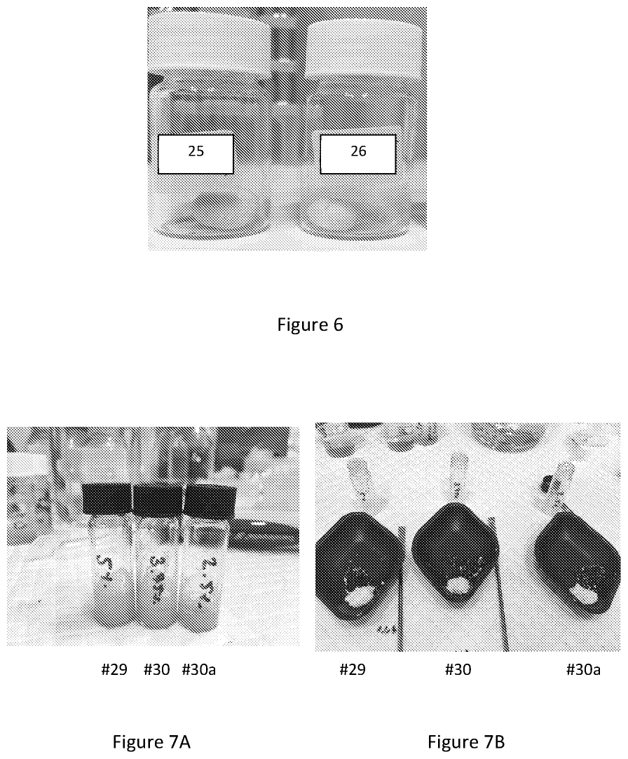

Putties containing PFD5 sodium, a tetracycline antibiotic, tricalcium phosphate and CMC were prepared (Table 5 and FIG. 6). First, the peptide, TCP and CMC powders were mixed followed by their hydration with an acid solution possibly supplemented with minocycline. 2% w/v of CMC was found to afford a putty with good malleability. The base to acid mole ratios can range from 1:4 to 1:0.17. Without being bound by any theory or mechanism of action, it is contemplated that the presence of CMC and TCP affords the formation of putties in which the peptide is unprotonated in its entirety and binds CMC and TCP at a low acid content.

TABLE 5

| | | | | TCP | | | | | HCl from | | | Base: | |
|---|---|---|---|---|---|---|---|---|---|---|---|---|---|
| | | | | % w/v | | | Mino- | Mino- | Mino- | HCL | | acid | Putty |
| | Peptide | Peptide | TCP | of supple- | CMC | CMC | cycline | cycline | cycline | added | Volume | mole | assess- |
| # | mg | mM | mg | mentation | mg | % w/v | HCl mg | w/v % | mM | mM | mL | ratio | ment |
| #25 | 25 | 15 | 1200 | 120 | 70 | 7% | 20 | 2% | 40 | 10 | 1 | 1:1.85 | G |
| #26 | 12.5 | 15 | 600 | 120 | 10 | 2% | 10 | 2% | 20 | 15 | 0.5 | 1:1.7 | G |
| #26a | 37.5 | 22.5 | 1400 | 140 | 40 | 4% | 20 | 2% | 40 | 20 | 1 | 1:1.65 | G |
| #26b | 37.5 | 22.5 | 1400 | 140 | 40 | 4% | 20 | 2% | 40 | — | 1 | 1:0.82 | G |
| #26c | 37.5 | 22.5 | 1400 | 140 | 40 | 4% | | | | 55 | 1 | 1:1.5 | G |
| | | | | | | | | | | or | | | |
| | | | | | | | | | | 50 | | | |

G = good malleable putty

When Minocin® was used as the minocycline source, to supplement 2.5% PFD5, no hydrogel was formed (formulation #27). Without being bound by any theory or mechanism of action, it is believed that the presence of magnesium ions from the magnesium sulfate heptahydrate excipient interferes with hydrogel formation. Nonetheless, the formulations which formed emulsion-like solutions (FIG. 7A) was further supplemented with TPC or TCP and CMC to form putties formulations #28-#30a; Table 6 and FIG. 7B).

TABLE 6

| | | | TCP | | | | Mino- | | | | Base: | | |
|---|---|---|---|---|---|---|---|---|---|---|---|---|---|
| | | | % w/v of | | | Mino- | cycline + | Total | NaOH | | acid | | Putty |
| | Peptide | Peptide | supple- | CMC | CMC | cycline | magnesium | HCl | added | Volume | mole | | assess- |
| # | mg | mM | mentation | mg | % w/v | % w/v | mg | mM | mM | mL | ratio | pH | ment |
| #28 | 12.5 | 15 | 130 | 20 | 4% | 2 | 10 | 40 | 70 | 0.5 | 1:0.4 | 6.0 | G |
| #29 | 25 | 30 | 140 | — | | 2 | 10 | 40 | 70 | 0.5 | 1:0.35 | 6.1 | F |
| #30 | 18.7 | 22.5 | 140 | — | | 2 | 10 | 40 | 70 | 0.5 | 1:0.37 | 6.0 | F |
| #30a | 12.5 | 15 | 140 | — | | | 10 | 40 | 70 | 0.5 | 1:0.4 | 5.9 | F |

G = good;
F = Fair

Additional formulations were prepared to assess the contribution of CMC to putty formation and the ratio between TCP and the peptide (Tables 7 and 8, respectively).

### Tables 7 and 8:

| | | Powder | | Dissolving solution | | | | |
|---|---|---|---|---|---|---|---|---|
| | Peptide % | CMC % | TCP % | | | V | Base: | Putty |
| # | w/v mg | w/v mg | w/v supp. | materials | pH | [mL] | acid* | formation |
| #28 | 2.5% | 4% | 130 | Minocycline | 4.69 | 0.5 | 1:0.4 | G |
| | 14.6 | 20 | | 2%, NaOH | | | | |
| | | | | (140 μL 1M), | | | | |
| | | | | Mg²⁺ 5.38% | | | | |
| #31 | 2.5% | 4% | 130 | Minocycline | 4.68 | 0.5 | 1:0.63 | F |
| | 14.4 | 20 | | 2%, NaOH | | | | |
| | | | | (70 μL 1M) | | | | |
| #32 | 2.5% | 10% | 130 | Minocycline | 4.69 | 0.2 | 1:0.4 | G |
| | 5.8 | 20 | | 2%, NaOH | | | | |
| | | | | (140 μL 1M), | | | | |
| | | | | Mg²⁺ 5.38% | | | | |
| #33 | 2.5% | — | 130 | Minocycline | 4.69 | 0.5 | 1:0.4 | F |
| | 14.4 | | | 2%, NaOH | | | | |
| | | | | (140 μL 1M), | | | | |
| | | | | Mg²⁺ 5.38% | | | | |
| #34 | 2.5% | — | 130 | Minocycline | 4.68 | 0.5 | 1:0.63 | N |
| | 14.6 | | | 2%, NaOH | | | | |
| | | | | (70 μL 1M) | | | | |

G = good;
F = Fair;
N = No putty was formed
*For acid concentration of 40 mM

| | Powder | | | | | |
|---|---|---|---|---|---|---|
| | Peptide | CMC | TCP* | | | |
| | % w/v | % w/v | % w/v | Final | Base: | Putty |
| # | (mg) mM | (mg) | (mg) | pH | acid | formation |
| #35 | 3.75% | 4% | 130% | 6.06 | 1:0.38 | Putty, |
| | (21.5 mg) | (20 mg) | (325*2) | | | slightly wet |
| | 22 mM | | | | | |
| #36 | 3.75% | 4% | 140% | 5.86 | 1:0.37 | Putty, good |
| | (21.5 mg) | (20 mg) | (350*2) | | | |
| | 22 mM | | | | | |
| #37 | 3.1% | 4% | 130% | 5.72 | 1:0.39 | Putty |
| | (17.8 mg) | (20 mg) | (325*2) | | | |
| | 18.6 mM | | | | | |
| #38 | 3.1% | 4% | 140% | 5.69 | 1:0.39 | Putty |
| | (17.8 mg) | (20 mg) | (350*2) | | | |
| | 18.6 mM | | | | | |
| #39 | 2.5% | 4% | 130% | 5.61 | 1:0.40 | Putty |
| | (14.3 mg) | (20 mg) | (325*2) | | | |
| | 15 mM | | | | | |

The effect of TCP particles size on putty appearance and hand-malleability was further assessed (Table 9). A 1:1 mixture of particles sizes 125-250 μm and 250-500 μm was found to yield a good putty formulation.

TABLE 9

| | Powder | | | | | | | |
|---|---|---|---|---|---|---|---|---|
| | | | TCP | TCP | | | | |
| | Peptide | CMC | 125-250 | 250-500 | Dissolving solution | | | |
| | % w/v | % w/v | [mg] | [mg] | | V | Final | Putty |
| # | (mg) | (mg) | (% of TCP) | (% of TCP) | | [mL] | pH | formation |
| #40 | 3.75% | 4% | 0 | 700 | Minocycline+ | 0.5 | 5.91 | Sandy |
| | (21.5) | (20) | | (100%) | Mg²⁺ + | | | |
| | | | | | NaOH | | | |
| #41 | 3.75% | 4% | 100 | 600 | Minocycline+ | 0.5 | 5.7 | Sandy |
| | (21.5) | (20) | (15%) | (85%) | Mg²⁺ + | | | |
| | | | | | NaOH | | | |
| #42 | 3.75% | 4% | 200 | 500 | Minocycline+ | 0.5 | 5.73 | Sandy |
| | (21.5) | (20) | (29%) | (71%) | Mg²⁺ + | | | |
| | | | | | NaOH | | | |
| #43 | 3.75% | 4% | 350 | 350 | Minocycline+ | 0.5 | 5.71 | Good |
| | (21.5) | (20) | (50%) | (50%) | Mg²⁺ + | | | |
| | | | | | NaOH | | | |

### Example 3. Minocycline Release from Formulation #43

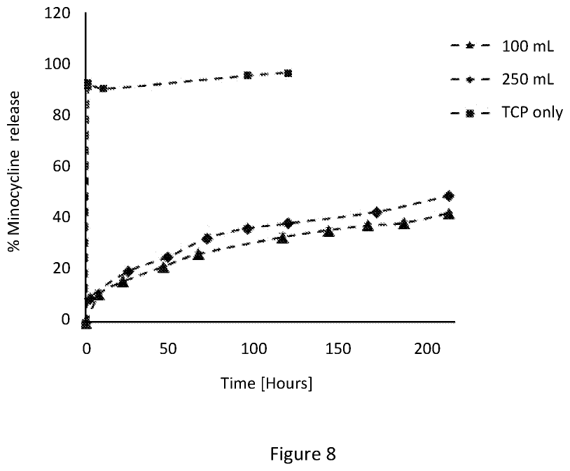

Minocycline-peptide formulation acts as a reservoir for sustained drug delivery of the tetracycline antibiotics. Formulation #43 was packed into a membrane and soaked into 250 ml or 100 ml HEPES buffer solution supplemented with 2.5 mM CaCl₂. Minocycline was released in a sustained manner (— 0.027 mg/h) over at least 9 days (FIG. 8). Since similar patterns were obtained for different volumes of buffer, it is deduced that ‘sink conditions’ were met. In comparison, TCP particles wetted with Minocin®-like solution released all minocycline in a couple of hours (FIG. 8).

### Example 4: Pre-Clinical Study of Periimplantitis

Bilateral extraction of the first mandibular molars of 18 rabbits is performed during the course of 30 days. Animals are divided at random into three groups. Immediately after removing the teeth, animals are treated as outlined in Table 10 below. The dosage depends on the size, shape and depth of the periodontal pockets. An average single dose of 0.25 g/pocket is the estimated dose. When the periodontal pocket is cleaned and dried, the putty is applied so that the entire pocket is filled with it. Excess putty is removed. The flap is then sutured in a simple, interrupted pattern.

TABLE 10

| Group | Treatment | | n | Dose |
|---|---|---|---|---|
| 1 | Standard of care | none | 3♀ + 3♂ | 0 |
| 2 | Composition with | ~0.25 g/ | 3♀ + 3♂ | ~17 mg peptide per 1 |
| | peptide and no | site × | | g putty |
| | API | 2 sites | | |
| 3 | Composition with | ~0.25 g/ | 3♀ + 3♂ | ~17 mg peptide & ~8 |
| | peptide and API | site × | | mg minocycline per 1 |
| | | 2 sites | | gram putty |

Evaluations includes:

Clinical Observations: Once daily during the 1^{st} week post-surgery and once weekly thereafter.

Implant site observation: As for clinical observations.

Mortality & Morbidity Check: At least once daily.

Body Weight: Prior to initiation, 2 days post-surgery, and once weekly thereafter.

Food Consumption: Once weekly.

Clinical Pathology: Hematology/Clinical Chemistry/Coagulation—Pre-dose, Days 7 & 30.

Ridge preservation by imaging (micro-CT): Following surgery, Days 7 & 30.

Histomorphometric analysis on bone core sample harvested on Day 30 (after imaging).

Blood collection for bioanalysis—pre-dose, Days 2, 7 & 30.

At study termination, animals are subjected to full necropsy. All organs are collected, weighed and fixed. Major organs are subjected to histopathological evaluation. All administration sites, from all animals are also collected and further subjected to histopathological and histomorphometric evaluations.

### Example 5. Clinical Study of Periimplantitis

Safety, tolerability, and efficacy of the compositions according to embodiments of the present invention in combination with mechanical debridement are evaluated in a randomized study in patients with periimplantitis as compared to mechanical debridement alone. A single treatment is performed immediately following debridement/standard of care. The primary efficacy measure is the reduction of probing depth at Day 180 as measured at qualifying implant sites. The inclusion criteria include adults ages 18 years or older having a minimum of one osseointegrated implant with a diagnosis of peri-implantitis and the absence of any other significant oral soft tissue pathology. Additionally, subjects included in the study have at least one peri-implant site with an average of 2 probing depth readings between 5 mm and 7 mm (inclusive) when using a light force with bleeding on probing within 30 seconds of the probing. Furthermore, subjects have a peri-implant intraosseous vertical defect with at least 3 mm defect depth as seen on an intraoral radiograph and during surgical exploration, an intraosseous component of at least 3 mm at the deepest point must be present. The primary safety end point is the incidence and severity of treatment-emergent (device related) adverse events (TEAEs) and the secondary safety end point is the number (%) of patients discontinued the study prematurely due to adverse events. The primary efficacy end point is the change in probing depth after 180 days as compared to baseline. The secondary efficacy end points are probing depth after 90 days, bleeding on probing after 90 and 180 days, changes in marginal bone level at dental implant after 90 and 180 days, bone in-growth after 90 and 180 days, infection non-recurrence after 3, 6, 12 m, serum/plasma antibiotics on Days 2, 7, 14, and 30. The exploratory end points are device absorption after 3, 6, 12 m and plaque levels and inflammatory response at 14, 30, 90 and 180 days vs. baseline. Monitoring is performed during 12 months.

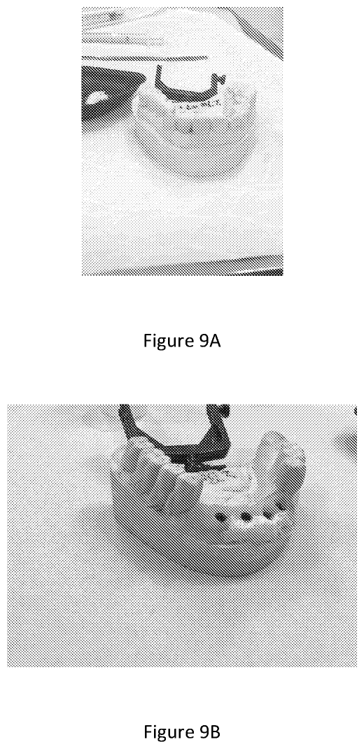

An immunology clinical study is performed using sera from human subjects implanted with compositions according to embodiments of the present invention. An average of 1.0 cc (range of 0.5 to 1.5 cc) of a composition according to embodiments of the present invention is implanted in 10 subjects as seen in FIG. 9A-9B. Sera is collected at the following time points: preoperatively (within 8 weeks of the surgery) and postoperatively at 2, 6, 12, 26, and 52 weeks. An electrochemiluminescence (ECL)-based assay for antibodies against the peptides of the present invention in human sera is evaluated to ensure that no immunogenic response to the peptides is elicited.

### Example 6. Hydrogels with NAC

Hydrogels according to certain embodiments of the present invention were prepared as follows: A first fraction was prepared by mixing 10-40 mg of the sodium salt of PFDS (SEQ ID NO: 1), and optionally drug (doxycycline 10 mg), NAC, carboxy methyl cellulose (CMC), and/or NaOH to obtain a solution or dispersion. The first fraction was sterilized using filtration or by autoclave and lyophilized to yield a powder. A second fraction containing water for injection or blood and derivatives thereof and optionally NAC solution (0.8-8%), minocycline (2%), and/or NaOH was prepared. The hydrogel was reconstituted by mixing the first and second fractions. Exemplary hydrogels are outlined in Tables 11-19 below:

TABLE 11
Hydrogel containing 2.5% PFD5, 0.8% NAC, and 1% doxycycline,
base:acid ~1:4.4 (NAC is considered as an acid)

| First Fraction | | | Second Fraction for Reconstitution | |
|---|---|---|---|---|
| Ingredient | Amount | | Ingredient | Amount |
| PFD5 | 25 | mg | Water for injection | 1 ml |
| Doxycycline | 10 | mg | | |
| NAC | 8 | mg | | |
| CMC | 37.5 | mg | | |
| NaOH (8 mM) | 5 | μl | | |

TABLE 12
Hydrogel containing 2.5% PFD5, and 5% NAC base:acid ~1:1

| | |
|---|---|
| First Fraction | Second Fraction for Reconstitution |

| Ingredient | Amount | Ingredient | Amount |
|---|---|---|---|
| PFD5 | 25 mg | NAC | 50 mg 1 ml |
| CMC | 37.5 mg | NaOH (290 mM) | |

TABLE 13
Hydrogel containing 2.5% PFD5, and 5% NAC, base:acid ~1:10

| | |
|---|---|
| First Fraction | Second Fraction for Reconstitution |

| Ingredient | Amount | | Ingredient | Amount |
|---|---|---|---|---|
| PFD5 | 25 | mg | Water for injection | 1 ml |
| NAC | 50 | mg | | |
| CMC | 37.5 | mg | | |
| NaOH (1M) | 12.3 | μl | | |

TABLE 14
Hydrogel containing 2.5% PFD5, 0.8% NAC,
and 2% minocycline, base:acid ~1:6

| First Fraction | | | Second Fraction for Reconstitution | | |
|---|---|---|---|---|---|
| Ingredient | Amount | | Ingredient | Amount | |
| PFD5 | 25 | mg | NAC | 8 | mg |
| CMC | 37.5 | mg | Minocycline | 20 | mg |
| NaOH (10 mM) | 4.5 | μl | Water for injection | 1 | ml |

TABLE 15
Hydrogel containing 4% PFD5, and 0.8% NAC, base:acid ~1:2

| First Fraction | | | Second Fraction for Reconstitution | |
|---|---|---|---|---|
| Ingredient | Amount | | Ingredient | Amount |
| PFD5 | 40 | mg | Water for injection or blood | 1 ml |
| NAC | 8 | mg | and derivatives thereof | |
| CMC | 37.5 | mg | | |
| NaOH (2.5 mM) | 10 | μl | | |

TABLE 16
Hydrogel containing 4% PFD5, and 0.8% NAC, base:acid ~1:1

| | |
|---|---|
| First Fraction | Second Fraction for Reconstitution |

| Ingredient | Amount | Ingredient | Amount |
|---|---|---|---|
| PFD5 | 40 mg | NAC | 8 mg |
| CMC | 37.5 mg | NaOH (25 mM) | 1 ml |

TABLE 17
Hydrogel containing 1% PFD5, and 8% NAC, base:acid ~1:66

| First Fraction | | | Second Fraction for Reconstitution | |
|---|---|---|---|---|
| Ingredient | Amount | | Ingredient | Amount |
| PFD5 | 10 | mg | Water for injection or blood | 1 ml |
| NAC | 80 | mg | and derivatives thereof | |
| CMC | 37.5 | mg | | |
| NaOH (90-100 mM) | 5 | μl | | |

TABLE 18
Hydrogel containing 1% PFD5, and 8% NAC, base:acid ~1:1

| | |
|---|---|
| First Fraction | Second Fraction for Reconstitution |

| Ingredient | Amount | Ingredient | Amount | |
|---|---|---|---|---|
| PFD5 | 10 mg | NAC | 80 | mg |
| CMC | 37.5 mg | NaOH (500 mM) | 1 | ml |

TABLE 19
Hydrogel containing 3.75% PFD5, and 5% NAC

| | | | Base:acid* | | Gel |
|---|---|---|---|---|---|
| Peptide | NAC | NaOH | molar ratio | pH | assessment |
| 22.5 mM | 312 mM | 265 mM | ~1:1 | 5.89 | Good |
| 3.75% | 5% | | | | |

*NAC is considered as an acid

### Example 7. Putties

Putties according to certain embodiments of the present invention were prepared as follows: A first fraction was prepared by mixing 10-40 mg of the sodium salt of PFDS (SED ID NO: 1), 37.5 mg carboxy methyl cellulose (CMC) and optionally NAC (8-80 mg), API (doxycycline 10 mg or minocycline 20 mg) and NaOH to obtain a solution or dispersion. The solution or dispersion was then sterilized. Sterile tricalcium phosphate (TCP: 1,300-1,500 mg) was then added followed by lyophilization to yield a powder. Alternatively, PFDS, CMC and TCP were sterilized by autoclave. A second fraction containing water for injection or blood and derivatives thereof, NAC solution (0.8-8%) optionally containing minocycline (2%) and NaOH was prepared. The putty was reconstituted by mixing the first and second fractions. Exemplary putties are outlined in Tables 20-28 below:

TABLE 20
Putty containing 2.5% PFD5, 8 mg NAC, and 10 mg doxycycline

| First Fraction | | | Second Fraction for Reconstitution | |
|---|---|---|---|---|
| Ingredient | Amount | | Ingredient | Amount |
| PFD5 | 25 | mg | Water for injection or blood | 1 ml |
| Doxycycline | 10 | mg | and derivatives thereof | |
| NAC | 8 | mg | | |
| CMC | 37.5 | mg | | |
| NaOH (4-5 mM) | 5 | μl | | |
| TCP | 1400 | mg | | |

TABLE 21
Putty containing 2.5% PFD5, 20 mg minocycline, and 10 mg NAC

| First Fraction | | | Second Fraction for Reconstitution | |
|---|---|---|---|---|
| Ingredient | Amount | | Ingredient | Amount |
| PFD5 | 25 | mg | Water for injection | 1 ml |
| minocycline | 20 | mg | | |
| NAC | 10 | mg | | |
| CMC | 37.5 | mg | | |
| NaOH (8-10 mM) | 5 | μl | | |
| TCP | 1500 | mg | | |

TABLE 22
Putty containing 2.5% PFD5, 20 mg minocycline, and 8 mg NAC

| | |
|---|---|
| First Fraction | Second Fraction for Reconstitution |

| Ingredient | Amount | Ingredient | Amount | |
|---|---|---|---|---|
| PFD5 | 25 mg | NAC | 8 | mg |
| CMC | 37.5 mg | Minocycline | 20 | mg |
| TCP | 1500 mg | NaOH (30 mM) | 1 | ml |

TABLE 23
Putty containing 2.5% PFD5, and 50 mg NAC

| | |
|---|---|
| First Fraction | Second Fraction for Reconstitution |

| Ingredient | Amount | Ingredient | Amount | |
|---|---|---|---|---|
| PFD5 | 25 mg | NAC | 50 | mg |
| CMC | 37.5 mg | NaOH (275-290 mM) | 1 | ml |
| TCP | 1500 mg | | | |

TABLE 24
Putty containing 4% PFD5, and 80 mg NAC

| | |
|---|---|
| First Fraction | Second Fraction for Reconstitution |

| Ingredient | Amount | Ingredient | Amount | |
|---|---|---|---|---|
| PFD5 | 40 mg | NAC | 80 | mg |
| CMC | 37.5 mg | WFI | 1 | ml |
| NaOH | 18.5 mg | | | |
| TCP | 1300 mg | | | |

TABLE 25
Putty containing 4% PFD5, and 50 mg NAC

| | |
|---|---|
| First Fraction | Second Fraction for Reconstitution |

| Ingredient | Amount | Ingredient | Amount | |
|---|---|---|---|---|
| PFD5 | 40 mg | NAC | 50 | mg |
| CMC | 37.5 mg | NaOH (275 mM) | 1 | ml |
| TCP | 1300 mg | | | |

TABLE 26
Putty containing 1% PFD5, and 80 mg NAC

| First Fraction | | | Second Fraction for Reconstitution | |
|---|---|---|---|---|
| Ingredient | Amount | | Ingredient | Amount |
| PFD5 | 10 | mg | Water for injection or blood | 1 ml |
| NAC | 80 | mg | and derivatives thereof | |
| CMC | 37.5 | mg | | |
| NaOH (95-100 mM) | 5 | μl | | |
| TCP | 1500 | mg | | |

TABLE 27
Putty containing 3.75% PFD5, and 5% NAC

| | | | | | Base:acid* | Putty |
|---|---|---|---|---|---|---|
| | | | | | molar | assess- |
| Peptide | NAC | NaOH | TCP | CMC | ratio | ment |
| 22.5 mM | 312 mM | 260 mM | 14 mM | 4% | ~1:1 | Very |
| 3.75% | 5% | | 140% | | | good |

*NAC is considered as an acid

TABLE 28
Putty containing 3.75% PFD5, and 3.97% NAC

| | | | | | Base:acid* | Putty |
|---|---|---|---|---|---|---|
| | | | | | molar | assess- |
| Peptide | NAC | NaOH | TCP | CMC | ratio | ment |
| 22.5 mM | 248 mM | 206 mM | 14 mM | 4% | ~1:1 | Very |
| 3.75% | 3.97% | | 140% | | | good |

*NAC is considered as an acid

### Example 8. Effect of NAC on Hydrogel Stabilization

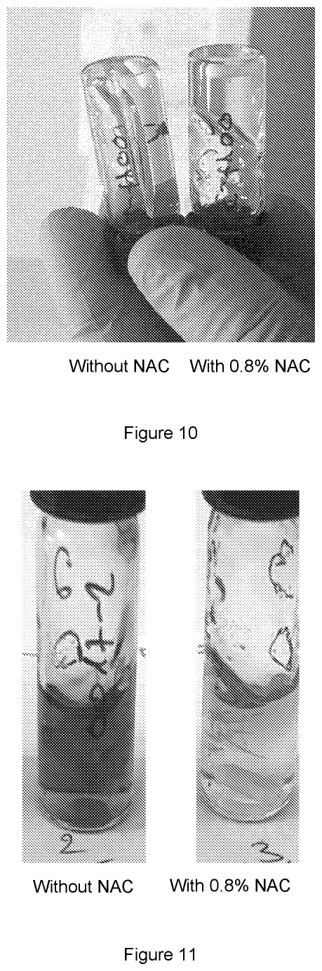

In order to evaluate the effect of NAC on the stability and consistency of a hydrogel formed from PFDS peptide (SED ID NO: 1), 2.5% hydrogels were prepared with or without NAC as outlined in Table 29 below. Gel that contained as low as 0.8% NAC was significantly more viscous than gel that did not contain NAC, although both gels had the same amount of peptide and the same pH (FIG. 10).

TABLE 29
Hydrogels containing 2.5% PFD5,
and 1% doxycycline with/out NAC

| | Hydrogel with NAC | Hydrogel without NAC |
|---|---|---|
| PFD5 [%] | 2.5 | 2.5 |
| NAC [%] | 0.8 | 0 |
| Doxycycline [%] | 1 | 1 |
| HCl [mM] | 0 | 10 |
| NaOH [mM] | 40 | 0 |
| pH | 6.30 | 6.25 |

The gels were then placed at room temperatures for 4 days. FIG. 11 shows that while a sample that contained NAC remained yellowish, a sample that was devoid of NAC became dark thereby indicating oxidation of the tetracycline antibiotics. Thus, low concentrations of NAC were effective in reducing tetracycline oxidation when compared to a NAC-free hydrogel.

The rheological properties of hydrogels with and without NAC (Table 30) were also determined using dynamic frequency sweep rheology measurements performed on an AR 2000 controlled stress rheometer (TA Instruments, New Castle, Del.) with the following system parameters:

Geometry: cone and plate mode with a cone angle of 4° and 20 mm diameter (for a volume of 150 μL).

Frequency: 0.5-50 rad/s.

Temp: 25° C.

Parameter: G′ and G″ elastic and loss moduli, respectively.

TABLE 30

| | Hydrogel with NAC | Hydrogel without NAC |
|---|---|---|
| PFD5 [%] | 2.5 | 2.5 |
| NAC [%] | 0.8 | 0 |
| HCl [mM] | 0 | 30 |
| NaOH [mM] | 19 | 0 |
| pH | 6.35 | 6.30 |

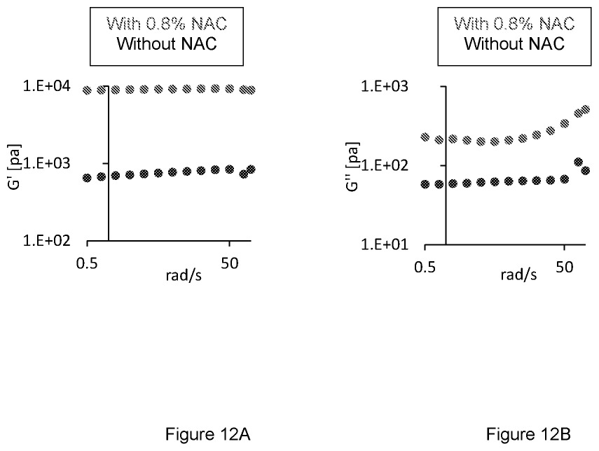

While both formulations resulted in clear and soft hydrogels, the rheological properties shown in FIGS. 12A and 12B clearly indicate that the presence of NAC resulted in significantly higher G′ and G″ values, compared to the hydrogel with no NAC.

The effect of NAC on the formation of hydrogel using three concentrations of peptide were also studied. Specifically, formulations with NAC (Tables 31-33) and without NAC (Table 34) were prepared and evaluated for hydrogel formation and clarity. While no hydrogel was formed even at high concentrations of peptide (4%) in water (Table 34, FIG. 13A), the addition of 0.8% NAC resulted in gelation even at lower concentrations of the peptide (2.5%; FIG. 13B). Thus, it is concluded that NAC is capable of stabilizing gel formation.

TABLE 31

| | | NAC 0.8% | | | |
|---|---|---|---|---|---|
| % | Peptide | solution in | | Hydrogel | |
| Peptide | [mg] | DDW [ml] | pH | Formation* | Clarity** |
| 1 | 27.2 | 2.366 | 4.08 | − | − |
| 2.5 | 61.4 | 2.137 | NA | +/− | +/− |
| 4 | 109 | 2.371 | 6.48 | + | + |

*(+) indicates that gel was formed and (-) indicates that no gel was formed
**(+) indicates a clear solution or hydrogel and (-) indicates a cloudy, non-homogeneous hydrogel.

TABLE 32

| | | NAC 0.8% | | Hydrogel | |
|---|---|---|---|---|---|
| % | Peptide | in NaOH | | Forma- | |
| Peptide | [mg] | solution [ml] | pH | tion* | Clarity** |
| 1 | 25.6 | 2.23 (37 mM NaOH) | 6.29 | − | + |
| 2.5 | 61.3 | 2.13 (19 mM NaOH) | 6.35 | + | + |
| 4 | 98.3 | 2.14 (1 mM NaOH) | 6.07 | + | + |

*(+) indicates that gel was formed and (−) indicates that no gel was formed
**(+) indicates a clear solution or hydrogel and (−) indicates a cloudy, non-homogeneous hydrogel.

TABLE 33

| | | NAC 5% | | Hydrogel | |
|---|---|---|---|---|---|
| % | Peptide | in NaOH | | Forma- | Clar- |
| Peptide | [mg] | solution [ml] | pH | tion* | ity** |
| 2.5 | 29.7 | 1.033 (290 mM NaOH) | 6.61 | + | + |

* (+) indicates that gel was formed and (−) indicates that no gel was formed
** (+) indicates a clear solution or hydrogel and (−) indicates a cloudy, non-homogeneous hydrogel.

TABLE 34

| % Peptide (titrated | | | | | |
|---|---|---|---|---|---|
| with HCl to pH 6 | | | | | |
| prior to | Peptide | DDW | | Hydrogel | |
| lyophilization) | [mg] | [ml] | pH | Formation* | Clarity** |
| 1 | 25.4 | 2.210 | 6.24 | − | + |
| 2.5 | 67.2 | 2.339 | 6.64 | − | + |
| 4 | 95.6 | 2.079 | 6.68 | − | + |

*(+) indicates that gel was formed and (−) indicates that no gel was formed
**(+) indicates a clear solution or hydrogel and (−) indicates a cloudy, non-homogeneous hydrogel.

### Example 9. Binding to Metals and Metal Oxides

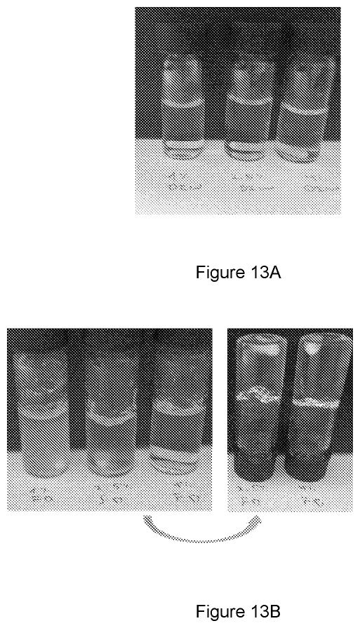

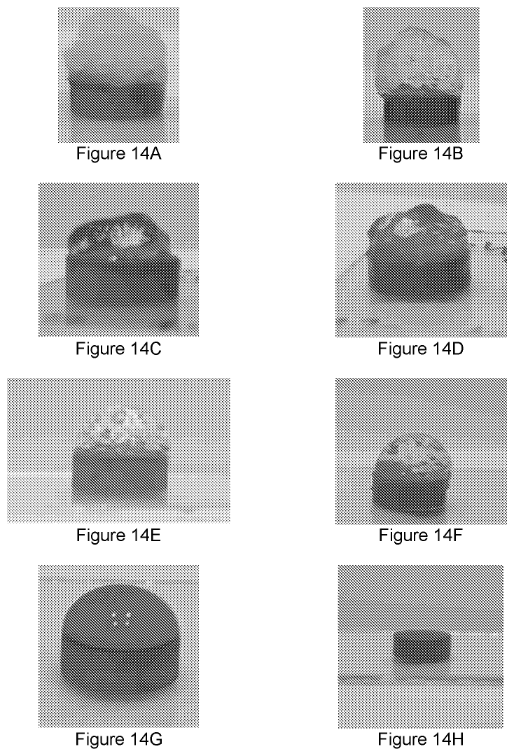

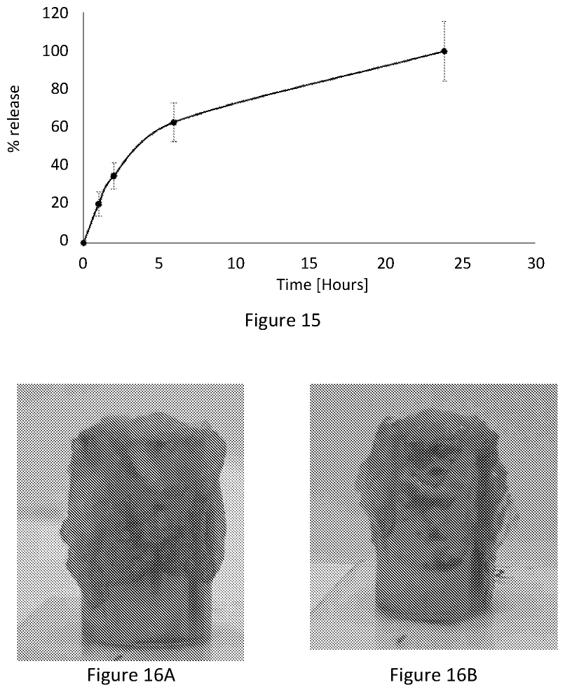

The binding capabilities of hydrogels according to embodiments of the present invention to metals and metal oxides were evaluated. Hydrogels containing PFDS peptide (SED ID NO: 1) at 2.5% (w/v) with 2% (w/v) of vancomycin and without vancomycin were applied to titanium discs. The gels were incubated at room temperature in a buffer containing 20 mM HEPES+2.5 mM CaCl₂, for up to 7 days. 2.5% (w/v) hyaluronic acid hydrogels were used as control. FIG. 14G-14H show that while 92% of the hyaluronic acid hydrogels dissolved after 1 hour, the hydrogels according to embodiments of the present invention adhered to the titanium discs up to 7 days following application with approximately 10% swelling after 1-2 days (FIG. 14A-14F). FIG. 15 shows the release profile of vancomycin from hydrogels according to embodiments of the present invention which were applied to titanium discs. Release was obtained for 25 hours following application.

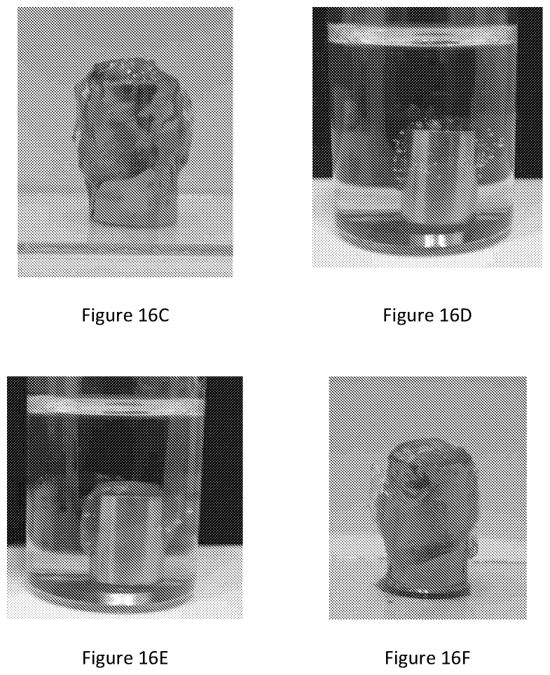

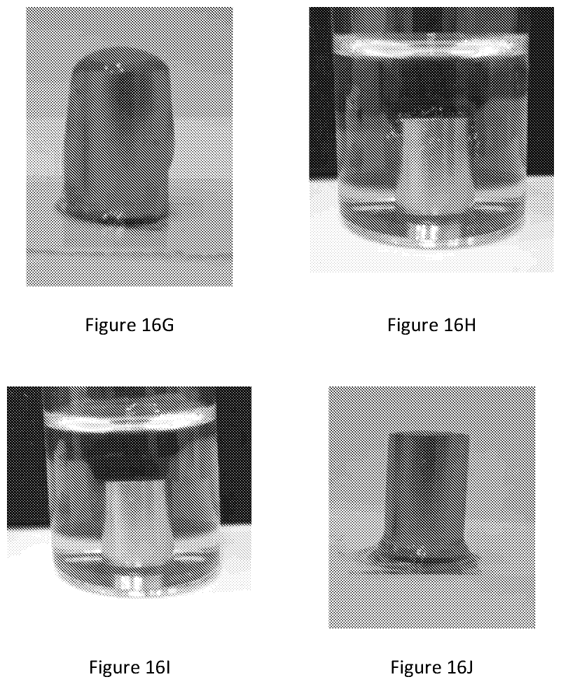

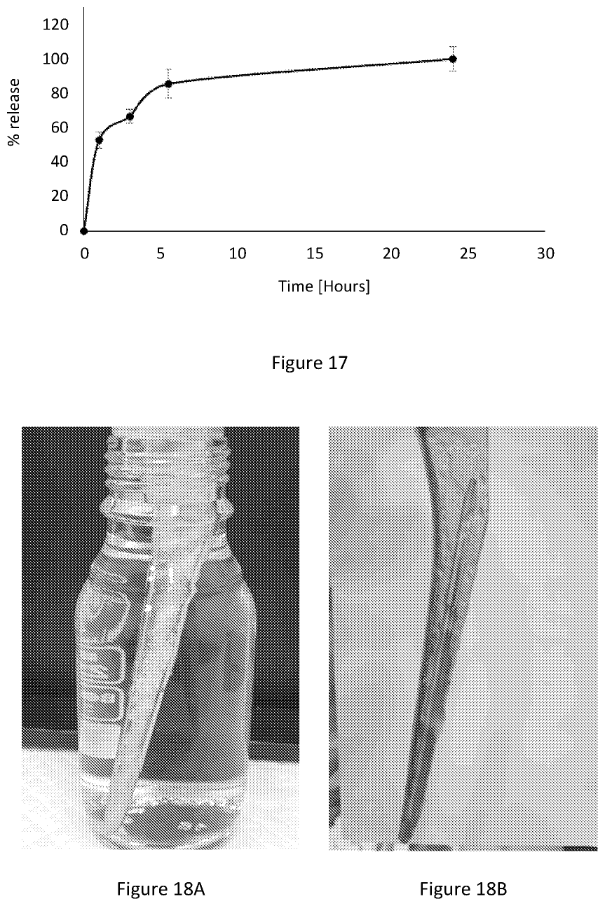

Hydrogels' adherence to Co Cr cylinders was further examined. FIG. 16A-16F show that while good adherence was obtained for the PFDS hydrogels, hydrogels based on hyaluronic acid failed to adhere to the Co Cr cylinders (FIG. 16G-16J). FIG. 17 shows the release profile of vancomycin from hydrogels according to embodiments of the present invention which were applied to Co Cr cylinders. Release was obtained for 25 hours following application.

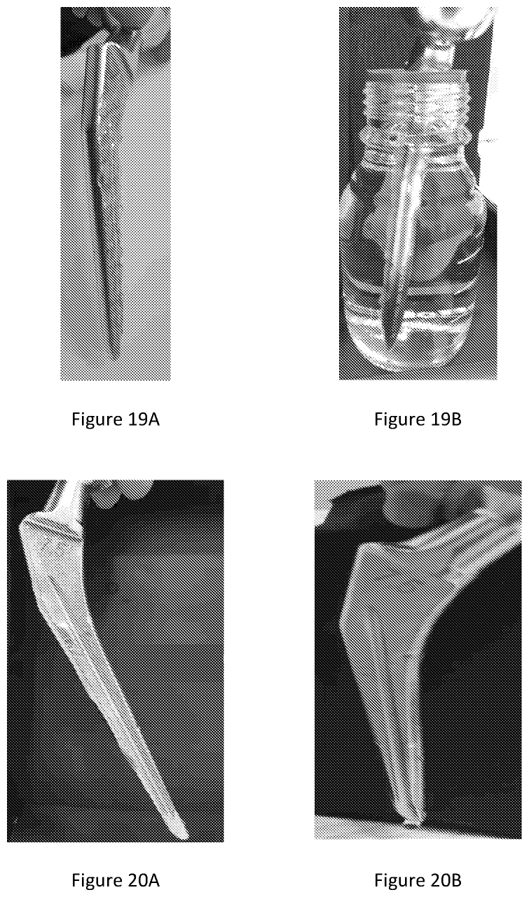

PFDS hydrogel with and without vancomycin were applied to a stainless steel implant and submerged in a buffer for 3 days and 1 day, respectively. FIGS. 18A-18B and 19A-19B show good adherence of the hydrogels to the implant. In contrast, hyaluronic acid hydrogel dissolved after 1 hour in a buffer with no apparent adherence to the stainless steel implant (FIG. 20A-20B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1-50. (canceled)

51. A pharmaceutical composition comprising:
(i) at least one amphiphilic peptide comprising alternating hydrophobic/hydrophilic amino acid residues, or a derivative or a salt thereof, wherein the peptide comprises 2-20 pairs of hydrophobic-hydrophilic alternating amino acid residues wherein the hydrophilic amino acid residue is selected from the group consisting of a negatively charged amino acid, a hydroxyl-containing amino acid, and a phosphorylated-hydroxyl-containing amino acid, and wherein said peptide has no more than 10% positively charged amino acid residues, and
(ii) an antibiotic selected from the group consisting of a tetracycline, a penicillin, a cephalosporin, an aminoglycozide, a glycopeptide, chloramphenicol, a quinolone, a sulphonamide, 5-nitroimidazole, an ansamycin, a macrolide, and a mixture or combination thereof.

52. The pharmaceutical composition of claim 51, wherein the at least one amphiphilic peptide has 4 to 40 amino acids; or wherein the at least one amphiphilic peptide comprises at least one terminal Pro residue; or wherein the hydrophobic amino acid residue is selected from the group consisting of Phe, Leu, Ile, Trp, Val, Trp, and Ala; or wherein the hydrophilic amino acid residue is selected from the group consisting of Glu, Asp, Tyr, Ser, Thr, Ser(PO₄), Thr(PO₄), and Tyr(PO₄).

53. The pharmaceutical composition of claim 51, wherein the at least one amphiphilic peptide comprises an amino acid sequence according to Formula I:
X-(hydrophobic-hydrophilic)ₙ-B (Formula I), or a pharmaceutically acceptable salt thereof,
wherein n designates an integer of 2 to 20, hydrophobic designates a hydrophobic amino acid residue, hydrophilic designates a hydrophilic amino acid residue, X designates Pro, Pro-hydrophilic or the peptide's amino terminus, and B designates Pro or the peptide's carboxy terminus.

54. The pharmaceutical composition of claim 53, wherein the at least one amphiphilic peptide comprises an amino acid sequence of any of the following formulae X-(Phe-Glu)ₙ-B, X-(Phe-Asp)ₙ-B, X-(Leu-Glu)ₙ-B, and X-(Leu-Asp)ₙ-B, or a pharmaceutically acceptable salt thereof.

55. The pharmaceutical composition of claim 53, wherein the at least one amphiphilic peptide comprises at least one modification selected from a modification of the amino terminus X and a modification of the carboxy terminus B, wherein the modification comprises acetylation of the amino terminus, amidation of the carboxy terminus, or a combination thereof.

56. The pharmaceutical composition of claim 51, wherein the at least one amphiphilic peptide comprises a sequence selected from the group consisting of:
(SEQ ID NO: 1)
Pro-(Asp-Phe)₅-Asp-Pro,
(SEQ ID NO: 2)
Pro-Glu-(Phe-Glu)₅,
(SEQ ID NO: 3)
Glu-(Phe-Glu)₅-Pro,
(SEQ ID NO: 4)
Pro-(Ser-Phe)₅-Ser-Pro,
(SEQ ID NO: 5)
Pro-(SerPO₄-Phe)₅-SerPO₄-Pro,
(SEQ ID NO: 6)
Pro-(TyrPO₄-Phe)₅-TyrPO₄-Pro,
(SEQ ID NO: 7)
Pro-(Glu-Leu)₅-Glu-Pro,
(SEQ ID NO: 8)
Pro-(Asp-Leu)₅-Asp-Pro,
(SEQ ID NO: 9)
Pro-(Ser-Leu)₅-Ser-Pro,
(SEQ ID NO: 10)
Pro-(SerPO₄-Leu)₅-SerPO₄-Pro,
(SEQ ID NO: 11)
Pro-(TyrPO₄-Leu)₅-TyrPO₄-Pro,
(SEQ ID NO: 12)
Pro-(Glu-Phe-Ser-Phe)₄-Glu-Pro,
(SEQ ID NO: 13)
Pro-(SerPO₄-Phe-Ser-Phe)₄-Ser-Pro,
(SEQ ID NO: 14)
Pro-(SerPO₄-Phe-Glu-Phe)₄-Glu-Pro,
(SEQ ID NO: 15)
Pro-(SerPO₄-Phe-Asp-Phe)₄-Asp-Pro,
(SEQ ID NO: 16)
Ala-Leu-Glu-(Phe-Glu)₃-Pro-Ala-(Glu-Phe)₃-Glu-Leu-
Pro-Ala-Leu-Glu-(Phe-Glu)₃-Pro,
(SEQ ID NO: 17)
Pro-Glu-(Phe-Glu)₂-Lys-(Glu-Phe)₂-Glu-Pro,
(SEQ ID NO: 18)
Pro-Glu-(Phe-Glu)₅-(Gly)₃-Arg-Gly-Asp-Ser,
(SEQ ID NO: 19)
(Phe-Glu)₃-Pro-(Gly)₃-Arg-Gly-Asp-Ser,
(SEQ ID NO: 20)
Ac-Pro-Asp-(Phe-Asp)₅-Pro-NH₂,
(SEQ ID NO: 21)
Pro-Asp-(Phe-Asp)₆,
(SEQ ID NO: 22)
(Phe-Asp)₆,
(SEQ ID NO: 23)
Pro-Glu-(Phe-Glu)₅-Pro,
(SEQ ID NO: 24)
Pro-Asp-(Phe-Asp)₅-Pro-NH₂,
(SEQ ID NO: 25)
(Phe-Glu)₅,
(SEQ ID NO: 26)
(Phe-Glu)₆,
(SEQ ID NO: 27)
(Phe-Glu)₇,
(SEQ ID NO: 28)
Pro-Asp-(Phe-Asp)₄,
(SEQ ID NO: 29)
Pro-Asp-(Phe-Asp)₆,
(SEQ ID NO: 30)
Pro-Asp-(Phe-Asp)₈,
(SEQ ID NO: 31)
(Phe-Asp)₅,
(SEQ ID NO: 32)
(Phe-Asp)₆,
(SEQ ID NO: 33)
(Phe-Asp)₇,
(SEQ ID NO: 34)
Pro-Asp-(Phe-Asp)₅-Pro-Arg-Gly-Asp-Ser,
(SEQ ID NO: 35)
Pro-(Phe-Asp)₃-Pro,
and
(SEQ ID NO: 36)
Pro-(Phe-Asp)₃-Pro-(Gly)₃-Arg-Gly-Asp-Ser,
or a pharmaceutically acceptable salt thereof.

57. The pharmaceutical composition of claim 56, wherein the at least one amphiphilic peptide comprises a sequence as set forth in SEQ ID NO: 1, or a pharmaceutically acceptable salt thereof.

58. The pharmaceutical composition of claim 51, wherein the antibiotic is a tetracycline antibiotic.

59. The pharmaceutical composition of claim 58, wherein the tetracycline antibiotic is selected from the group consisting of chlortetracycline, oxytetracycline, demeclocycline, doxycycline, lymecycline, meclocycline, methacycline, minocycline, rolitetracycline, chlorotetracycline, tigecycline, and a pharmaceutically acceptable salt thereof.

60. The pharmaceutical composition of claim 51, wherein the antibiotic is a combination of minocycline and rifampicin, or pharmaceutically acceptable salts thereof; or wherein the antibiotic is a combination of vancomycin and rifampicin, or pharmaceutically acceptable salts thereof.

61. The pharmaceutical composition of claim 51, wherein the antibiotic is an aminoglycoside; or wherein the antibiotic is a glycopeptide.

62. The pharmaceutical composition of claim 61, wherein the aminoglycoside is selected from the group consisting of kanamycin A, amikacin, tobramycin, dibekacin, gentamicin, sisomicin, netilmicin, neomycins B, C or E, streptomycin, and a pharmaceutically acceptable salt thereof; or wherein the glycopeptide is vancomycin or a pharmaceutically acceptable salt thereof.

63. The pharmaceutical composition of claim 51 further comprising at least one pharmaceutically acceptable excipient.

64. The pharmaceutical composition of claim 63, wherein the pharmaceutically acceptable excipient comprises at least one of a chelating agent, a buffering or pH adjusting agent, a preservative, an anti-oxidant, a thickening agent, a tonicity enhancing agent, a tissue adhesive, an inorganic mineral, and a mixture or combination thereof.

65. The pharmaceutical composition of claim 64, wherein the chelating agent is selected from the group consisting of disodium edetate, deferoxamine mesylate (desferrioxamine), 2,3-dimercaprol, meso-2,3-dimercaptosuccinic acid (DMSA) and its ester analogues, deferiprone, nitrilotriacetic acid (NTA), and a mixture or combination thereof; or wherein the buffering or pH adjusting agent is selected from the group consisting of sodium hydroxide, potassium hydroxide, arginine, lysine, hydrochloric acid, aspartic acid, glutamic acid, and a mixture or combination thereof; or wherein the anti-oxidant comprises at least one of ascorbic acid, N-acetyl cysteine (NAC), derivatives and salts thereof; or wherein the thickening agent comprises at least one of hydroxypropyl methylcellulose, hydroxypropyl cellulose, hydroxyethyl cellulose, hydroxymethyl cellulose, carboxy methyl cellulose, polyvinylpyrrolidone, polyvinyl alcohol, and a mixture or combination thereof; or wherein the inorganic mineral comprises at least one of hydroxyapatite, calcium phosphate, calcium carbonate, calcium gluconate, calcium oxalate, calcium sulfate, calcium chloride, magnesium phosphate, magnesium carbonate, magnesium gluconate, magnesium oxalate, magnesium sulfate, magnesium chloride, zinc phosphate, zinc carbonate, zinc gluconate, zinc oxalate, zinc sulfate, zinc chloride, sodium bicarbonate, and a mixture or combination thereof.

66. The pharmaceutical composition of claim 51 further comprising at least one of an antiresorptive agent and an anti-inflammatory agent.

67. A method of coating an implant, the method comprising the step of applying a therapeutically effective amount of a pharmaceutical composition according to claim 51 to the implant prior to implantation, wherein the implant is a metal implant, a metal oxide implant or a ceramic implant.

68. A method of treating or preventing a disease or disorder associated with mineralized tissue, the method comprising administering to a subject in need thereof a therapeutically effective amount of a pharmaceutical composition according to claim 51, wherein the disease or disorder is selected from the group consisting of a subchondral bone lesion, osteomyelitis, peri-prosthetic joint infection, and surgery site infection.

69. A method of treating or preventing an infection in a subject having a primary joint replacement, a revision joint replacement, avascular necrosis, a high-risk contralateral hip fracture, a delayed union fracture, a non-union fracture, an open fracture, a cartilage transplant, or a stress fracture, the method comprising administering to a subject in need thereof a therapeutically effective amount of a pharmaceutical composition according to claim 51.

70. A method of treating or preventing a progressive periodontal disease, the method comprising administering to a subject in need thereof a therapeutically effective amount of a pharmaceutical composition according to claim 51, wherein the progressive periodontal disease is selected from periodontitis and periimplantitis.
